(19) United States
(12) Patent Application Publication
(10) Pub. No.: US 2024/0293277 A1
(43) Pub. Date: Sep. 5, 2024

(54) SYSTEM FOR LOCATING PATIENT SUPPORT APPARATUSES

(71) Applicant: Stryker Corporation, Kalamazoo, MI (US)

(72) Inventors: Kirby M. Neihouser, Portage, MI (US); Krishna Sandeep Bhimavarapu, Kalamazoo, MI (US); Celso Henrique Farnese Pires Pereira, Portage, MI (US); Madhu Sandeep Thota, Portage, MI (US); Jerald A. Trepanier, Augusta, MI (US); Madhu Thomas, London, Ontario (CA); Thomas Deeds, Seattle, WA (US); Marco Constant, Portage, MI (US)

(21) Appl. No.: 18/572,678

(22) PCT Filed: Sep. 15, 2022
(86) PCT No.: PCT/US2022/043585

§ 371 Date: Dec. 20, 2023

Related U.S. Application Data
(60) Provisional application No. 63/245,245, filed on Sep. 17, 2021.
(60) Provisional application No. 63/323,202, filed on Mar. 24, 2022.

(51) Int. Cl. A61G 7/05; G16H 40/67

(52) U.S. Cl. CPC A61G 7/05; G16H 40/67; A61G 2203/40; A61G 2205/60

(57) ABSTRACT

A patient support apparatus for supporting a patient is adapted to wirelessly communicate with one or more wall units. A controller onboard the patient support apparatus and/or a controller onboard the wall unit is adapted to use RF signals to determine a distance between the wall unit and the patient support apparatus. In some embodiments, the wall unit and/or patient support apparatus include an orientation sensor adapted to determine the geographic orientation of the wall unit and/or patient support apparatus. The patient support apparatus controller may use the geographic orientation information to determine if the wall unit is facing toward the patient support apparatus or not. The patient support apparatus may communicate with multiple wall units concurrently, and/or the wall unit may communicate with multiple patient support apparatuses concurrently.

### BACKGROUND

The present disclosure relates to patient support apparatuses, such as beds, cots, stretchers, recliners, or the like. More specifically, the present disclosure relates to patient support apparatuses that communicate with one or more wall units that are affixed to the walls of a healthcare facility at known locations.

The administrators of healthcare facilities typically wish to know the location of each of the patient support apparatuses within their facility. In the past, the locations of patient support apparatuses have been determined in different manners. In some systems, patient support apparatuses have been configured to communicate with headwall units when the patient support apparatuses are aimed at, and positioned within close proximity to, the headwall unit. The headwall unit provides a unique identifier to the patient support apparatus that can be correlated to a unique location within the healthcare facility. The patient support apparatus is only able to communicate with the headwall unit when it is in close proximity because it uses infrared communications that require line-of-sight communication, and because the headwall units are designed to have a short range (e.g. about five feet or so). Because of this short range communication, the location of the patient support apparatus can be concluded to be within that same short range of the headwall unit when it is communicatively paired with the headwall unit.

### SUMMARY

According to the various aspects described herein, the present disclosure is directed to a location detection system that overcomes past issues and/or provides improved functionality with respect to prior location determining systems. In one aspect, the system enables patient support apparatuses to communicate with one or more wall units regardless of the orientation of the patient support apparatus relative to the wall unit. In another aspect, the patient support apparatuses are able to concurrently communicate with multiple wall units and/or the wall units are able to concurrently communicate with multiple patient support apparatuses, thereby enabling a single wall unit to provide location information to multiple patient support apparatuses and/or enabling a single patient support apparatus to gain location information from multiple wall units. In still other aspects, electromagnetic shielding and/or beamforming techniques may be utilized by the wall units in order to help the patient support apparatuses better distinguish between wall units that are placed in close proximity to each other. Still other improvements and/or advantages over prior art location systems will become apparent in light of the following written description and the accompanying drawings.

According to a first aspect of the present disclosure, a patient support apparatus is provided that includes a support surface, a first transceiver, a second transceiver, and a controller. The support surface is adapted to support a patient thereon. The first transceiver is coupled to a first location on the patient support apparatus and is adapted to wirelessly communicate with a wall unit mounted to a wall of a healthcare facility in which the patient support apparatus is positioned. The second transceiver is adapted to communicate with a network of the healthcare facility. The controller is adapted to use radio frequency (RF) communications between the wall unit and the first transceiver to determine a distance between the wall unit and the first transceiver.

According to another aspect of the present disclosure, a patient support apparatus is provided that includes a support surface, a first transceiver, an orientation sensor, and a controller.

In some aspects, the controller is further adapted to receive a unique identifier from the wall unit and to forward the unique identifier to the network using the second transceiver.

The controller, in some aspects, determines the distance without using any infrared signals.

In some aspects, the first transceiver is further adapted to wirelessly communicate with a second wall unit mounted to a second wall of the healthcare facility, and the controller is further adapted to use RF communications between the second wall unit and the first transceiver to determine a second distance between the second wall unit and the first transceiver.

The first transceiver may be an ultra-wideband transceiver.

According to some aspects, the first transceiver is adapted to wirelessly communicate with the wall unit regardless of an orientation of the patient support apparatus with respect to the wall.

The patient support apparatus, in some aspects, further includes a third transceiver and a memory. The third transceiver is coupled to a second location on the patient support apparatus and is adapted to communicate using RF signals with the wall unit. The memory contains data identifying a relative position of the third transceiver with respect to the first transceiver.

In some aspects, the patient support apparatus further includes a fourth transceiver coupled to a third location on the patient support apparatus and adapted to communicate using RF signals with the wall unit. The memory further contains data identifying relative positions of the fourth transceiver with respect to the first and third transceivers.

In some aspects, the controller is further adapted to determine an orientation of the patient support apparatus with respect to the wall to which the wall unit is mounted.

The controller, in some aspects, is further adapted to use RF communications between the wall unit and the third transceiver to determine a second distance between the wall unit and the third transceiver; and to use RF communications between the wall unit and the fourth transceiver to determine a third distance between the wall unit and the fourth transceiver.

The first, third, and fourth transceivers, in some embodiments, are further adapted to communicate with a tag attached to a mobile medical device, and the controller is further adapted to use the first, third, and fourth transceivers to determine a relative position of the mobile medical device with respect to the patient support apparatus.

The controller may be further adapted to associate the mobile medical device with the patient support apparatus if the mobile medical device is positioned within a volume of space defined around the patient support apparatus.

In some aspects, the patient support apparatus further includes an orientation sensor adapted to determine a first geographical orientation of the patient support apparatus. The controller may be adapted receive a second geographical orientation of the wall unit and to use the first and second geographical orientations to determine if the patient support apparatus and the wall unit are facing toward each other or not.

The patient support apparatus may further comprise a Bluetooth transceiver adapted to communicate with the wall unit using Bluetooth signals.

When a Bluetooth transceiver is included, the controller may be further adapted to transmit audio signals from a microphone onboard the patient support apparatus to the wall unit using the Bluetooth transceiver.

The orientation sensor, in some aspects, is a magnetometer.

According to another aspect of the present disclosure, a wall unit is provided that is adapted to be mounted to a wall of a healthcare facility. The wall unit includes a first transceiver and a controller. The first transceiver is adapted to wirelessly communicate with a patient support apparatus and the controller is adapted to use radio frequency (RF) signals between the patient support apparatus and the first transceiver to determine a distance between the patient support apparatus and the first transceiver.

According to still another aspect of the present disclosure, a wall unit is provided that is adapted to be mounted to a wall of healthcare facility. The wall unit includes a first transceiver adapted to wirelessly communicate with a patient support apparatus, an orientation sensor adapted to determine a geographical orientation of the wall unit, and a controller adapted transmit the geographical orientation of the wall unit to the patient support apparatus using the first transceiver.

According to other aspects of the present disclosure, the controller is further adapted to forward the distance to the patient support apparatus.

In some aspects, the wall unit includes a memory in which is stored a geographical orientation of the wall unit, and the controller is adapted to transmit the geographical orientation of the wall unit to the patient support apparatus.

The wall unit, in some aspects, further includes a shielding layer positioned between the first transceiver and the wall unit. The shielding layer is adapted to attenuate the RF signals emitted from the first transceiver that travel toward the wall.

The shielding layer may include a mu metal.

In some aspects, the wall unit further includes a nurse call interface adapted to electrically couple to a wall outlet mounted in the wall. The nurse call interface includes a plurality of first pins adapted to electrically couple to a plurality of second pins defined in the wall outlet when the nurse call interface is coupled to the wall outlet, and the controller is adapted to change an electrical state of first one of the plurality of first pins in response to a message received from the patient support apparatus.

In some aspects, the wall unit includes a Bluetooth transceiver adapted to communicate with the patient support apparatus using Bluetooth signals.

The controller, in some aspects, is further adapted to receive audio signals via the Bluetooth transceiver from the patient support apparatus and to transfer the audio signals to a second one of the plurality of first pins.

The first transceiver, in some aspects, is further adapted to concurrently wirelessly communicate with a second patient support apparatus, and the controller is further adapted to use RF communications between the second patient support apparatus and the wall unit to determine a second distance between the patient support apparatus and the first transceiver.

The controller, in some aspects, is further adapted to forward the second distance to the second patient support apparatus.

The wall unit, in some aspects, further includes a second transceiver and a memory. The second transceiver is adapted to wirelessly communicate with the patient support apparatus. The memory contains data identifying a relative position of the first transceiver with respect to the second transceiver. The controller is further adapted to use RF signals between the patient support apparatus and the second transceiver to determine a second distance between the patient support apparatus and the second transceiver.

In some aspects, the controller is further adapted to forward the second distance to the patient support apparatus.

The wall unit, in some aspects, includes an infrared transceiver adapted to detect an infrared interrogation signal from the patient support apparatus. The controller may be further adapted to respond to the infrared interrogation signal by transmitting a unique identifier of the wall unit to the patient support apparatus using the infrared transceiver.

The controller of the wall unit, in some aspects, is further adapted to use RF signals between the first transceiver and a tag attached to a mobile medical device to determine a second distance between the first transceiver and the tag. The controller may be further adapted to forward the second distance to the patient support apparatus.

The orientation sensor may be a magnetometer.

Before the various aspects of the disclosure are explained in detail, it is to be understood that the claims are not to be limited to the details of operation or to the details of construction and the arrangement of the components set forth in the following description or illustrated in the drawings. The aspects described herein are capable of being practiced or being carried out in alternative ways not expressly disclosed herein. Also, it is to be understood that the phraseology and terminology used herein are for the purpose of description and should not be regarded as limiting. The use of “including” and “comprising” and variations thereof is meant to encompass the items listed thereafter and equivalents thereof as well as additional items and equivalents thereof. Further, enumeration may be used in the description of various embodiments. Unless otherwise expressly stated, the use of enumeration should not be construed as limiting the claims to any specific order or number of components. Nor should the use of enumeration be construed as excluding from the scope of the claims any additional steps or components that might be combined with or into the enumerated steps or components.

### BRIEF DESCRIPTION OF THE DRAWINGS

FIG. 1 is a perspective view of a patient support apparatus according to a first aspect of the present disclosure;

FIG. 2 is a plan view of an illustrative caregiver control panel of the patient support apparatus of FIG. 1;

FIG. 3 is a plan view of an illustrative patient control panel of the patient support apparatus of FIG. 1;

FIG. 4 is a perspective view of the patient support apparatus and a first type of wall unit that is used for automatically detecting the location of a patient support apparatus;

FIG. 5 is a block diagram of the patient support apparatus, wall unit, and network of FIG. 4;

FIG. 6 is a perspective view of the patient support apparatus and a second type of wall unit that is used for automatically detecting the location of the patient support apparatus;

FIG. 7 is a block diagram of the patient support apparatus, wall unit, and network of FIG. 6;

FIG. 8 is a diagram of a wall unit and patient support apparatus that include orientation sensors and/or orientation data;

FIG. 9 is a diagram of a patient support apparatus with multiple location sensors showing an example of how the patient support apparatus is able to determine which side of a wall it is positioned on;

FIG. 10 is a diagram of a patient support apparatus shown positioned within an association threshold of a linked wall unit;

FIG. 11 is a diagram of a pair of wall units that both include electromagnetic shielding;

FIG. 12 is a diagram of a plurality of patient support apparatuses that are shown communicating with a plurality of wall units in a one-to-one manner;

FIG. 13 is a diagram of a plurality of patient support apparatuses that are each shown communicating with a single wall unit in a many-to-one manner;

FIG. 14 is a diagram of a patient support apparatus adapted to determine its location via a wall unit and to additionally determine the location of a mobile medical device; and

FIG. 15 is a diagram of an alternative second type of wall unit that is used for automatically detecting the position of one or more devices used in the care of a patient.

### DETAILED DESCRIPTION OF THE EMBODIMENTS

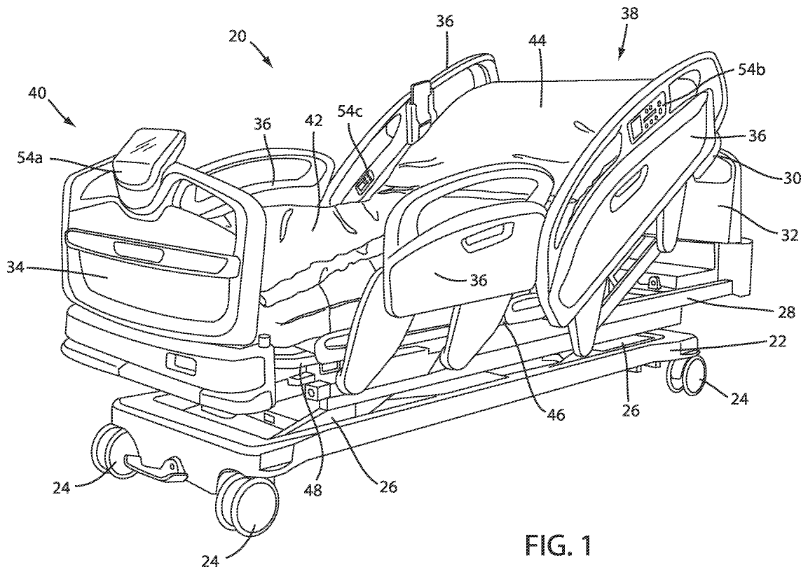

An illustrative patient support apparatus 20 according to an embodiment of the present disclosure is shown in FIG. 1. Although the particular form of patient support apparatus 20 illustrated in FIG. 1 is a bed adapted for use in a hospital or other medical setting, it will be understood that patient support apparatus 20 could, in different embodiments, be a cot, a stretcher, a recliner, an operating table, or any other structure capable of supporting a patient in a healthcare environment.

In general, patient support apparatus 20 includes a base 22 having a plurality of wheels 24, a pair of lifts 26 supported on the base 22, a litter frame 28 supported on the lifts 26, and a support deck 30 supported on the litter frame 28. Patient support apparatus 20 further includes a headboard 32, a footboard 34 and a plurality of siderails 36. Siderails 36 are all shown in a raised position in FIG. 1 but are each individually movable to a lower position in which ingress into, and egress out of, patient support apparatus 20 is not obstructed by the lowered siderails 36.

Lifts 26 are adapted to raise and lower litter frame 28 with respect to base 22. Lifts 26 may be hydraulic actuators, electric actuators, or any other suitable device for raising and lowering litter frame 28 with respect to base 22. In the illustrated embodiment, lifts 26 are operable independently so that the tilting of litter frame 28 with respect to base 22 can also be adjusted, to place the litter frame 28 in a flat or horizontal orientation, a Trendelenburg orientation, or a reverse Trendelenburg orientation. That is, litter frame 28 includes a head end 38 and a foot end 40, each of whose height can be independently adjusted by the nearest lift 26. Patient support apparatus 20 is designed so that when an occupant lies thereon, his or her head will be positioned adjacent head end 38 and his or her feet will be positioned adjacent foot end 40.

Litter frame 28 provides a structure for supporting support deck 30, the headboard 32, footboard 34, and siderails 36. Support deck 30 provides a support surface for a mattress 42, or other soft cushion, so that a person may lie and/or sit thereon. In some embodiments, the mattress 42 includes one or more inflatable bladders that are controllable via a blower, or other source of pressurized air. In at least one embodiment, the inflation of the bladders of the mattress 42 is controllable via electronics built into patient support apparatus 20. In one such embodiments, mattress 42 may take on any of the functions and/or structures of any of the mattresses disclosed in commonly assigned U.S. Pat. No. 9,468,307 issued Oct. 18, 2016, to inventors Patrick Lafleche et al., the complete disclosure of which is incorporated herein by reference. Still other types of mattresses may be used.

Support deck 30 is made of a plurality of sections, some of which are pivotable about generally horizontal pivot axes. In the embodiment shown in FIG. 1, support deck 30 includes at least a head section 44, a thigh section 46, and a foot section 48, all of which are positioned underneath mattress 42 and which generally form flat surfaces for supporting mattress 42. Head section 44, which is also sometimes referred to as a Fowler section, is pivotable about a generally horizontal pivot axis between a generally horizontal orientation (not shown in FIG. 1) and a plurality of raised positions (one of which is shown in FIG. 1). Thigh section 46 and foot section 48 may also be pivotable about generally horizontal pivot axes.

In some embodiments, patient support apparatus 20 may be modified from what is shown to include one or more components adapted to allow the user to extend the width of patient support deck 30, thereby allowing patient support apparatus 20 to accommodate patients of varying sizes. When so modified, the width of deck 30 may be adjusted sideways in any increments, for example between a first or minimum width, a second or intermediate width, and a third or expanded/maximum width.

As used herein, the term “longitudinal” refers to a direction parallel to an axis between the head end 38 and the foot end 40. The terms “transverse” or “lateral” refer to a direction perpendicular to the longitudinal direction and parallel to a surface on which the patient support apparatus 20 rests.

It will be understood by those skilled in the art that patient support apparatus 20 can be designed with other types of mechanical constructions, such as, but not limited to, that described in commonly assigned, U.S. Pat. No. 10,130,536 to Roussy et al., entitled PATIENT SUPPORT USABLE WITH BARIATRIC PATIENTS, the complete disclosure of which is incorporated herein by reference. In another embodiment, the mechanical construction of patient support apparatus 20 may include the same, or nearly the same, structures as the Model 3002 S3 bed manufactured and sold by Stryker Corporation of Kalamazoo, Michigan. This construction is described in greater detail in the Stryker Maintenance Manual for the MedSurg Bed, Model 3002 S3, published in 2010 by Stryker Corporation of Kalamazoo, Michigan, the complete disclosure of which is incorporated herein by reference. In still another embodiment, the mechanical construction of patient support apparatus 20 may include the same, or nearly the same, structure as the Model 3009 Procuity MedSurg bed manufactured and sold by Stryker Corporation of Kalamazoo, Michigan. This construction is described in greater detail in the Stryker Maintenance Manual for the 3009 Procuity MedSurg bed (publication 3009-009-002, Rev. A.0), published in 2020 by Stryker Corporation of Kalamazoo, Michigan.

It will be understood by those skilled in the art that patient support apparatus 20 can be designed with still other types of mechanical constructions, such as, but not limited to, those described in commonly assigned, U.S. Pat. No. 7,690,059 issued Apr. 6, 2010, to Lemire et al., and entitled HOSPITAL BED; and/or commonly assigned U.S. Pat. publication No. 2007/0163045 filed by Becker et al, and entitled PATIENT HANDLING DEVICE INCLUDING LOCAL STATUS INDICATION, ONE-TOUCH FOWLER ANGLE ADJUSTMENT, AND POWER-ON ALARM CONFIGURATION, the complete disclosures of both of which are also hereby incorporated herein by reference. The overall mechanical construction of patient support apparatus 20 may also take on still other forms different from what is disclosed in the aforementioned references provided the patient support apparatus includes the functions and features discussed in greater detail below.

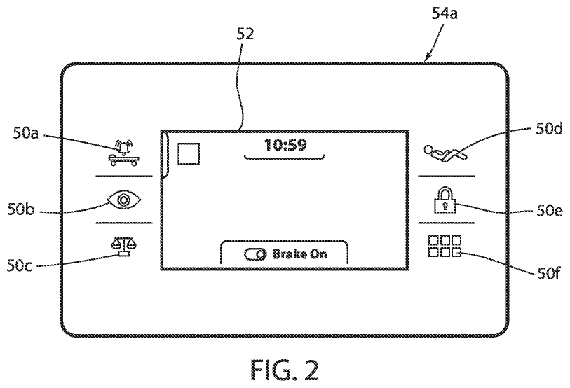

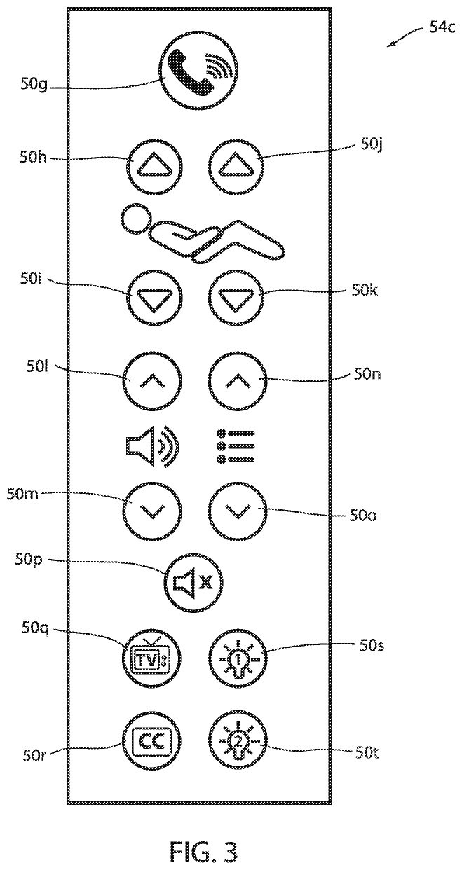

Patient support apparatus 20 further includes a plurality of control panels 54 that enable a user of patient support apparatus 20, such as a patient and/or an associated caregiver, to control one or more aspects of patient support apparatus 20. In the embodiment shown in FIG. 1, patient support apparatus 20 includes a footboard control panel 54a, a pair of outer siderail control panels 54b (only one of which is visible), and a pair of inner siderail control panels 54c (only one of which is visible). Footboard control panel 54a and outer siderail control panels 54b are intended to be used by caregivers, or other authorized personnel, while inner siderail control panels 54c are intended to be used by the patient associated with patient support apparatus 20. Each of the control panels 54 includes a plurality of controls 50 (see, e.g. FIGS. 2-3), although each control panel 54 does not necessarily include the same controls and/or functionality.

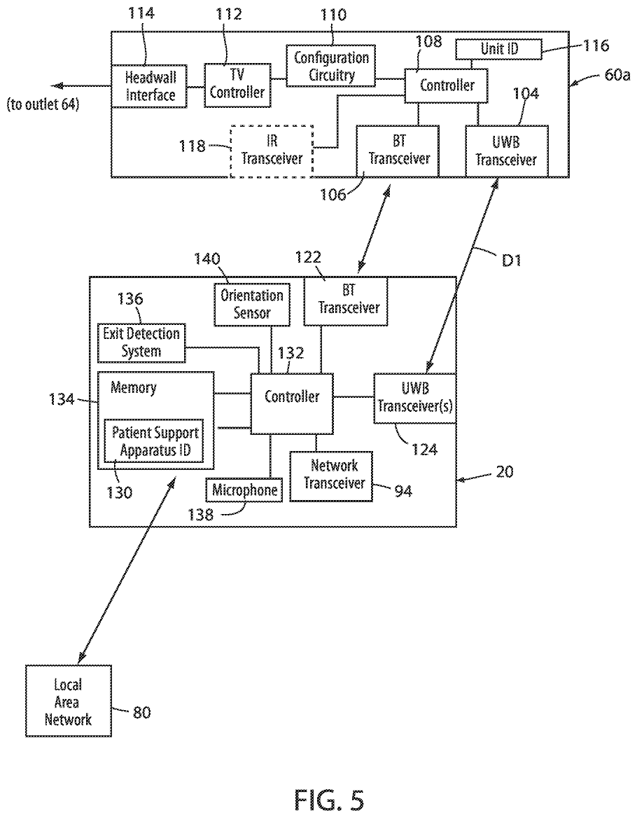

Among other functions, controls 50 of control panel 54a allow a user to control one or more of the following: change a height of support deck 30, raise or lower head section 44, activate and deactivate a brake for wheels 24, arm and disarm an exit detection system 136 (FIG. 5), change various settings on patient support apparatus 20, view the current location of the patient support apparatus 20 as determined by the location detection system discussed herein, view what medical devices—if any—the patient support apparatus 20 has associated itself with, and perform other actions. One or both of the inner siderail control panels 54c also include at least one control that enables a patient to call a remotely located nurse (or other caregiver). In addition to the nurse call control, one or both of the inner siderail control panels 54c also include one or more controls for controlling one or more features of one or more room devices positioned within the same room as the patient support apparatus 20. As will be described in more detail below, such room devices include, but are not necessarily limited to, a television, a reading light, and a room light. With respect to the television, the features that may be controllable by one or more controls 50 on control panel 54c include, but are not limited to, the volume, the channel, the closed-captioning, and/or the power state of the television. With respect to the room and/or night lights, the features that may be controlled by one or more controls 50 on control panel 54c include the on/off state and/or the brightness level of these lights.

Control panel 54a includes a display 52 (FIG. 2) configured to display a plurality of different screens thereon. Surrounding display 52 are a plurality of navigation controls 50a-f that, when activated, cause the display 52 to display different screens on display 52. More specifically, when a user presses navigation control 50a, control panel 54a displays an exit detection control screen on display 52 that includes one or more icons that, when touched, control an onboard exit detection system 136 (FIG. 5). The exit detection system 136 is as adapted to issue an alert when a patient exits from patient support apparatus 20. Exit detection system 136 may include any of the same features and functions as, and/or may be constructed in any of the same manners as, the exit detection system disclosed in commonly assigned U.S. patent application No. 62/889,254 filed Aug. 20, 2019, by inventors Sujay Sukumaran et al, and entitled PERSON SUPPORT APPARATUS WITH ADJUSTABLE EXIT DETECTION ZONES, the complete disclosure of which is incorporated herein by reference. Other types of exit detection systems may be included within patient support apparatus 20.

When a user presses navigation control 50b (FIG. 2), control panel 54 displays a monitoring control screen that includes a plurality of control icons that, when touched, control an onboard monitoring system built into patient support apparatus 20. Further details of one type of monitoring system that may be built into patient support apparatus 20 are disclosed in commonly assigned U.S. patent application Ser. No. 62/864,638 filed Jun. 21, 2019, by inventors Kurosh Nahavandi et al, and entitled PATIENT SUPPORT APPARATUS WITH CAREGIVER REMINDERS, as well as commonly assigned U.S. patent application Ser. No. 16/721,133 filed Dec. 19, 2019, by inventors Kurosh Nahavandi et al, and entitled PATIENT SUPPORT APPARATUSES WITH MOTION CUSTOMIZATION, the complete disclosures of both of which are incorporated herein by reference. Other types of monitoring systems may be included within patient support apparatus 20.

When a user presses navigation control 50c, control panel 54a displays a scale control screen that includes a plurality of control icons that, when touched, control the scale system of patient support apparatus 20. Such a scale system may include any of the same features and functions as, and/or may be constructed in any of the same manners as, the scale systems disclosed in commonly assigned U.S. patent application No. 62/889,254 filed Aug. 20, 2019, by inventors Sujay Sukumaran et al, and entitled PERSON SUPPORT APPARATUS WITH ADJUSTABLE EXIT DETECTION ZONES, and U.S. patent application Ser. No. 62/885,954 filed Aug. 13, 2019, by inventors Kurosh Nahavandi et al, and entitled PATIENT SUPPORT APPARATUS WITH EQUIPMENT WEIGHT LOG, the complete disclosures of both of which are incorporated herein by reference. The scale system may utilize the same force sensors that are utilized by the exit detection system 136, or it may utilize one or more different sensors. Other scale systems besides those mentioned above in the '254 and '954 applications may alternatively be included within patient support apparatus 20.

When a user presses navigation control 50d, control panel 54 displays a motion control screen that includes a plurality of control icons that, when touched, control the movement of various components of patient support apparatus 20, such as, but not limited to, the height of litter frame 28 and the pivoting of head section 44. In some embodiments, the motion control screen displayed on display 52 in response to pressing control 50d may be the same as, or similar to, the position control screen 216 disclosed in commonly assigned U.S. patent application Ser. No. 62/885,953 filed Aug. 13, 2019, by inventors Kurosh Nahavandi et al, and entitled PATIENT SUPPORT APPARATUS WITH TOUCHSCREEN, the complete disclosure of which is incorporated herein by reference. Other types of motion control screens may be included on patient support apparatus 20.

When a user presses navigation control 50e, control panel 54a displays a motion lock control screen that includes a plurality of control icons that, when touched, control one or more motion lockout functions of patient support apparatus 20. Such a motion lockout screen may include any of the features and functions as, and/or may be constructed in any of the same manners as, the motion lockout features, functions, and constructions disclosed in commonly assigned U.S. patent application Ser. No. 16/721,133 filed Dec. 19, 2019, by inventors Kurosh Nahavandi et al, and entitled PATIENT SUPPORT APPARATUSES WITH MOTION CUSTOMIZATION, the complete disclosure of which is incorporated herein by reference. Other types of motion lockouts may be included within patient support apparatus 20.

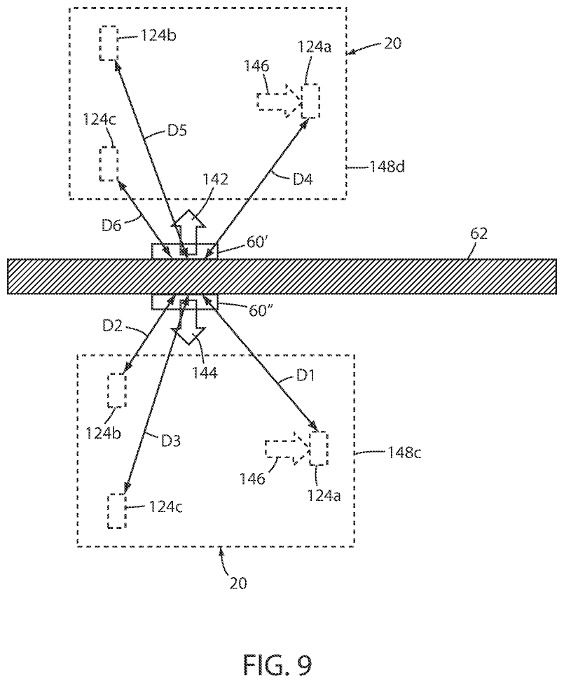

When a user presses on navigation control 50f, control panel 54a displays a menu screen that includes a plurality of menu icons that, when touched, bring up one or more additional screens for controlling and/or viewing one or more other aspects of patient support apparatus 20. Such other aspects include, but are not limited to, diagnostic and/or service information for patient support apparatus 20, mattress control and/or status information, configuration settings, location information, medical device association information, and other settings and/or information. One example of a suitable menu screen is the menu screen 100 disclosed in commonly assigned U.S. patent application Ser. No. 62/885,953 filed Aug. 13, 2019, by inventors Kurosh Nahavandi et al, and entitled PATIENT SUPPORT APPARATUS WITH TOUCHSCREEN, the complete disclosure of which is incorporated herein by reference. Other types of menus and/or settings may be included within patient support apparatus 20. In at least one embodiment, utilization of navigation control 50f allows a user to navigate to a screen that enables a user to configure the communication settings between patient support apparatus 20 and one or more wall units (discussed more below). Examples of the type of communication settings that may be configured in this manner are disclosed in, and illustrated in FIGS. 9-15 of, commonly assigned U.S. patent application Ser. No. 63/26,937 filed May 19, 2020, by inventors Alexander Bodurka et al, and entitled PATIENT SUPPORT APPARATUSES WITH HEADWALL COMMUNICATION, the complete disclosure of which is incorporated herein by reference.

For all of the navigation controls 50a-f (FIG. 2), screens other than the ones specifically mentioned above may be displayed on display 52 in other embodiments of patient support apparatus 20 in response to a user pressing these controls. Thus, it will be understood that the specific screens mentioned above are merely representative of the types of screens that are displayable on display 52 in response to a user pressing on one or more of navigation controls 50a-f. It will also be understood that, although navigation controls 50a-f have all been illustrated in the accompanying drawings as dedicated controls that are positioned adjacent display 52, any one or more of these controls 50a-f could alternatively be touchscreen controls that are displayed at one or more locations on display 52. Still further, although controls 50a-f have been shown herein as buttons, it will be understood that any of controls 50a-f could also, or alternatively, be switches, dials, or other types of non-button controls.

FIG. 3 illustrates one example of a patient control panel 54c that may be incorporated into patient support apparatus 20 and positioned at a location on patient support apparatus 20 that is convenient for a patient to access while supported on support deck 30, such as on an interior side of one of the siderails 36. Control panel 54c includes a plurality of controls 50g-t that are intended to be operated by a patient. A nurse call control 50g, when pressed by the patient, sends a signal to a nurse call system requesting that a remotely positioned nurse talk to the patient. A Fowler-up control 50h, when pressed by the patient, causes a motorized actuator onboard patient support apparatus 20 to raise Fowler section 44 upwardly. A Fowler-down control 50i, when pressed by the patient, causes the motorized actuator to lower Fowler section 44 downwardly. A gatch-up control 50j, when pressed by the patient, causes another motorized actuator to raise a knee section of support deck 30, while a gatch-down control 50k causes the motorized actuator to lower the knee section of support deck 30.

A volume-up control 50l, when pressed by the patient, causes patient support apparatus 20 to send a signal to an in-room television instructing it to increase its volume, while a volume down control 50m, when pressed, causes patient support apparatus 20 to send a signal to the television instructing it to decrease its volume. A channel-up control 50n, when pressed by the patient, causes patient support apparatus 20 to send a signal to the television instructing it to increase the channel number, while a channel-down control 500, when pressed, causes patient support apparatus 20 to send a signal to the television instructing it to decrease the channel number.

A mute control 50p, when pressed, causes patient support apparatus 20 to send a signal to the television instructing it to either mute itself or unmute itself, depending upon whether the television is currently muted or unmuted. In other words, mute control 50p is a toggle control that alternatingly sends mute and unmute commands to the television when it is pressed.

Power control 50q is a toggle control that, when pressed, sends a signal to the television to either turn on or turn off, depending upon the television's current power status. Closed-captioning control 50r is another toggle control that, when pressed, sends a signal to the television to either turn on its closed-captioning feature or to turn off its closed captioning feature, depending upon whether the closed-captioning feature is currently on or off.

Control 50s is a toggle control that, when pressed, sends a signal to a first light to either turn on or turn off, depending upon the current state of that first light. Control 50t is another toggle control that, when pressed, sends a signal to a second light to either turn on or turn off, depending upon the current state of that second light. In some embodiments, the first light is a reading light and the second light is a room light, both of which are positioned off-board the patient support apparatus 20.

It will be understood that not only the number of controls 50 on control panel 54c, but also the functions of the controls 50 on control panel 54c, the layout of the controls 50 on control panel 54c, and/or other aspects of control panel 54c may be modified from what is shown in FIG. 3. In some embodiments, control panel 54c is implemented on a pendant controller that includes a cable that is plugged into a port on patient support apparatus 20. In other embodiments, one or more of the controls 50 of control panel 54c may be omitted, augmented, and/or split amongst other controls panels and/or locations. Still other manners of implementing control panel 54c are also possible.

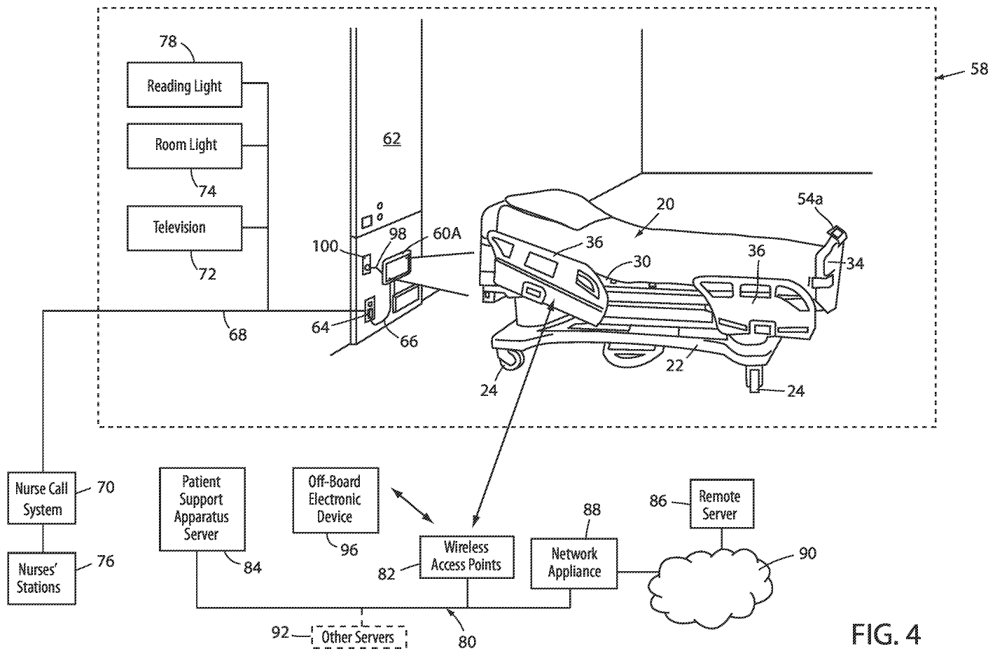

FIG. 4 illustrates a system for determining the location of patient support apparatus 20 when positioned within a room 58 of a conventional healthcare facility, such as, but not limited to, a hospital. This location detection system includes patient support apparatus 20, one or more wall units 60 (which may be of two types, 60a or 60b, as will be discussed in more detail below) and, in some aspects, a remote server, such as patient support apparatus server 84. Wall units 60 are positioned at known and fixed locations within the healthcare facility in which patient support apparatus 20 is positioned. As will be discussed in greater detail below, wall units 60 are adapted to determine how far away one or more patient support apparatus 20 are from the wall unit 60 and/or they are adapted to allow patient support apparatuses 20 to determine how far away they are positioned from the wall unit 60.

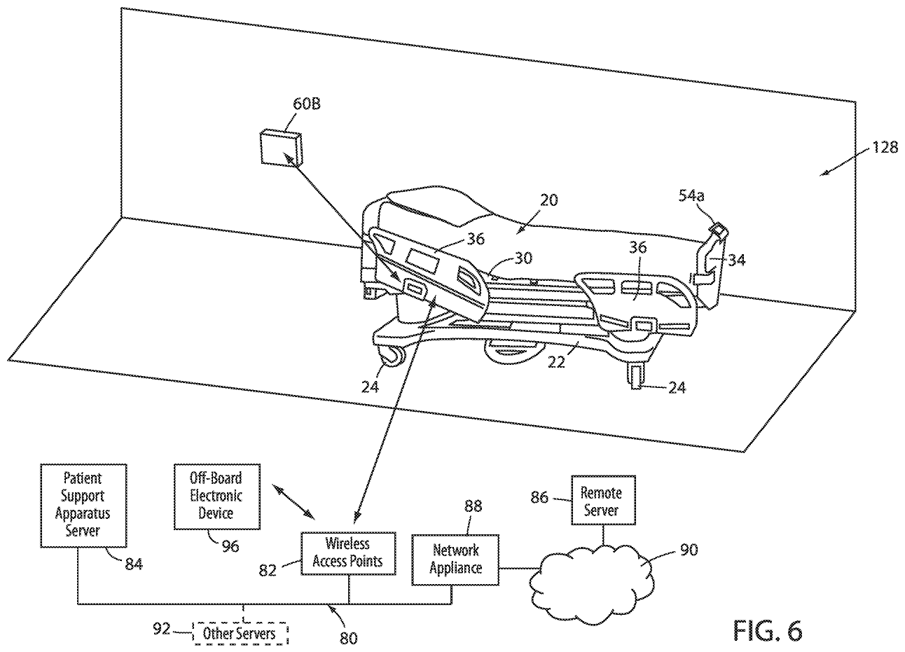

The location detection system described herein may utilize two different types of wall units 60: linked wall units 60a and unlinked wall units 60b. One example of a linked wall unit 60a is shown in FIG. 4. One example of an unlinked wall unit 60b is shown in FIG. 6. Wall units 60a and 60b differ from each other in that linked wall units 60a are adapted to communicate with a conventional communication outlet 64 that is typically built into one or more walls of a healthcare facility. That is, wall units 60a are communicatively linked to a conventional communication outlet 64. Unlinked wall units 60b are not adapted to communicate with such communication outlets 64, and are therefore not linked to a nearby communications outlet 64. Both wall units 60a and 60b are adapted to provide location information to a patient support apparatus. Linked wall units 60a, however, are also adapted to serve as a communication conduit for routing communications between patient support apparatus 20 and one or more devices and/or systems that are communicatively coupled to communication outlet 64 (e.g. room devices 72, 74, 78, and/or nurse call system 70). Unlinked wall units 60b, in contrast, are not necessarily adapted to serve as communication conduits between patient support apparatus 20 and any other electronic structures. In general, linked wall units 60a are typically positioned in patient rooms of the healthcare facility where one or more communication outlets 64 are typically present, while unlinked wall units 60b are typically positioned in locations outside of patient rooms, such as hallways, maintenance areas, and/or other areas. Unless explicitly stated otherwise, references to “wall units 60” made herein refer to both wall units 60a and 60b.

As shown in FIG. 4, linked wall units 60a are adapted to be mounted to a wall 62, such as a headwall of a patient room 58 within the healthcare facility. The headwall of a conventional healthcare facility room 58 typically includes a conventional communications outlet 64 physically integrated therein. Communications outlet 64 is adapted to receive a nurse call cable 66 that physically connects at its other end either to patient support apparatus 20 (not shown) or to wall unit 60a (shown in FIG. 4). In many healthcare facilities, communication outlet 64 includes a 37-pin connector, although other types of connectors are often found in certain healthcare facilities. As will be discussed in greater detail below, linked wall unit 60a and nurse call cable 66 allow patient support apparatus 20 to communicate with a nurse call system, and one or more room devices positioned within room 58.

Communication outlet 64 is electrically coupled to one or more cables, wires, or other conductors 68 that electrically couple the communication outlet 64 to a nurse call system 70 and one or more conventional room devices, such as a television 72, a room light 74, and/or a reading light 76. Conductors 68 are typically located behind wall 62 and not visible. In some healthcare facilities, conductors 68 may first couple to a room interface circuit board that includes one or more conductors 68 for electrically coupling the room interface circuit board to room device 72, 74, 76 and/or nurse call system 70. Still other communicative arrangements for coupling communication outlet 64 to nurse call system 70 and/or one or more room devices 72, 74, 76 are possible.

Nurse call cable 66 (FIG. 4) enables patient support apparatus 20 to communicate with nurse call system 70 and/or room devices 72, 74, 76. A patient supported on patient support apparatus 20 who activates a nurse call control (e.g. 50g; see FIG. 3) on patient support apparatus 20 causes a signal to be wirelessly sent from patient support apparatus 20 to linked wall unit 60a, which in turn conveys the signal via nurse call cable 66 to the nurse call system 70, which forwards the signal to one or more remotely located nurses (e.g. nurses at one or more nurse's stations 76). If the patient activates one or more room device controls (e.g. controls 50l-t; see FIG. 3), one or more wireless signals are conveyed to linked wall unit 60a, which in turn sends appropriate signals via nurse call cable 66 to communication outlet 64 and the room device 72, 74, 78 that change one or more features of these devices (e.g. the volume, channel, on/off state, etc.).

As is also shown in FIG. 4, patient support apparatus 20 is further configured to communicate with a local area network 80 of the healthcare facility. In the embodiment shown in FIG. 4, patient support apparatus 20 includes a wireless network transceiver 94 (FIG. 5) that communicates wirelessly with local area network 80. Network transceiver 94 is, in at least some embodiments, a WiFi transceiver (e.g. IEEE 802.11) that wirelessly communicates with one or more conventional wireless access points 82 of local area network 80. In other embodiments, network transceiver 94 may be a wireless transceiver that uses conventional 5G technology to communicate with network 80, one or more servers hosted thereon, and/or other devices. In some embodiments, network transceiver 94 may include any of the structures and/or functionality of the communication modules 56 disclosed in commonly assigned U.S. Pat. No. 10,500,401 issued to Michael Hayes and entitled NETWORK COMMUNICATION FOR PATIENT SUPPORT APPARATUSES, the complete disclosure of which is incorporated herein by reference. Still other types of wireless network transceivers may be utilized.

In some embodiments, network transceiver 94 is a wired transceiver that is adapted to allow patient support apparatus 20 to communicate with network 80 via a wired connection, such as an Ethernet cable that plugs into an Ethernet port (e.g. an RJ-45 style port, an 8P8C port, etc.) built into patient support apparatus 20. In still other embodiments, patient support apparatus 20 includes both a wired transceiver 94 for communicating with network 80 via a wired connection and a wireless transceiver 94 for wirelessly communicating with network 80.

Patient support apparatus 20 is configured to communicate with one or more servers on local area network 80 of the healthcare facility. One such server is a patient support apparatus server 84. Patient support apparatus server 84 is adapted, in at least one embodiment, to receive status information from patient support apparatuses 20 positioned within the healthcare facility and distribute this status information to caregivers, other servers, and/or other software applications. As will be discussed in greater detail below, server 84 may also be configured to receive data from one or more medical devices that are positioned within a volume of space defined around patient support apparatus 20. Alternatively, in some embodiments where data from medical devices is collected, the data from one or more of medical devices may be forwarded to one or more other servers 92 on network 80 (and/or one or more electronic devices 96), such as a caregiver assistance server and/or a caregiver assistance software application, as will also be discussed in greater detail below.

In some embodiments, patient support apparatus server 84 is configured to communicate at least some of the patient support apparatus status data and/or medical device data received from patient support apparatuses 20 to a remote server 86 that is positioned geographically remotely from the healthcare facility. Such communication may take place via a conventional network appliance 88, such as, but not limited to, a router and/or a gateway, that is coupled to the Internet 90. The remote server 86, in turn, is also coupled to the Internet 90, and patient support apparatus server 84 is provided with the URL and/or other information necessary to communicate with remote server 86 via the Internet connection between network 80 and server 86.

In some alternative embodiments, patient support apparatus 20 may be configured to communicate directly with one or more cloud-based servers, such as remote server 86, without utilizing patient support apparatus server 84. That is, in some embodiments, patient support apparatuses 20 may be configured to communicate directly with a remote server without relying upon any locally hosted servers (e.g. servers hosted on network 80). In one such embodiment, patient support apparatus 20 utilizes Microsoft's Azure could computing service to directly connect to one or more remote servers 86 without utilizing server 84. In some such embodiments, network appliance 88 is a router configured to support such direct connections. Still other types of direct-to-cloud connections may be utilized with one or more of patient support apparatuses 20.

Patient support apparatus server 84 is also configured to determine the location of each patient support apparatus 20, or receive the location of each patient support apparatus 20 from the patient support apparatuses 20. In some embodiments, patient support apparatus server 84 determines the room number and/or bay area of each patient support apparatus 20 that is positioned within a room 58, as well as the location of patient support apparatuses 20 that are positioned outside of a room 58, such as, those that may be positioned in a hallway, a maintenance area, or some other area. In general, patient support apparatus server 84 may be configured to determine the position of any patient support apparatus 20 that is positioned within communication range of one or more wall units 60, as will be discussed in greater detail below.

It will be understood that the architecture and content of local area network 80 will vary from healthcare facility to healthcare facility, and that the example shown in FIG. 4 is merely one example of the type of network a healthcare facility may be employ. Typically, one or more additional servers 92 will be hosted on network 80 and one or more of them may be adapted to communicate with patient support apparatus server 84. For example, an electronic health record server will typically be present in any healthcare facility, and in some embodiments discussed herein, it will be in communication with patient support apparatus server 84 in order to receive patient data that is to be recorded in a patient's health record (e.g. weight readings taken from the scales built into patient support apparatuses 20; therapies provided to patients using a powered mattress 42 onboard patient support apparatuses 20, data from one or more medical devices that are determined to be associated with the patient assigned to patient support apparatus 20, etc.). Local area network 80 will also typically allow one or more electronic devices 96 to access the local area network 80 via wireless access points 82. Such electronic devices 96 include, but are not limited to, smart phones, tablet computers, portable laptops, desktop computers, smart televisions, and other types of electronic devices that include a WiFi capability and that are provided with the proper credentials (e.g. SSID, password, etc.) to access network 80 (and, in at least some situations, patient support apparatus server 84).

Linked wall units 60a are adapted to wirelessly receive signals from patient support apparatus 20 and deliver the signals to communications outlet 64 in a manner that matches the way the signals would otherwise be delivered to communications outlet 64 if a conventional nurse call cable 66 were connected directly between patient support apparatus 20 and communications outlet 64. Linked wall units 60a are also adapted to transmit signals received from communications outlet 64 to patient support apparatus 20 via BT transceiver 106 and/or UWB transceiver 104. Thus, patient support apparatus 20 and linked wall unit 60a cooperate to send signals to, and receive signals from, communications outlet 64 in a manner that is transparent to communications outlet 64 such that outlet 64 cannot detect whether it is in communication with patient support apparatus 20 via a wired connection or it is in communication with patient support apparatus 20 via a wireless connection between patient support apparatus 20 and linked wall unit 60a (the latter of which is in wired communication with outlet 64). In this manner, a healthcare facility can utilize the wireless communication abilities of one or more patient support apparatuses 20 without having to make any changes to their existing communication outlets 64.

As noted, in addition to sending signals received from patient support apparatus 20 to communications outlet 64, linked wall units 60a are also adapted to forward signals received from communications outlet 64 to patient support apparatus 20. Linked wall units 60a are therefore adapted to provide bidirectional communication between patient support apparatus 20 and communications outlet 64. This bidirectional communication includes, but is not limited to, communicating command signals from any of controls 50 and/or from any of electronic devices 96 to corresponding room devices 72, 74, and/or 78 and communicating audio signals between a person supported on patient support apparatus 20 and a caregiver positioned remotely from patient support apparatus 20. The audio signals received by wall units 60 from a microphone on patient support apparatus 20 are forwarded to communications outlet 64 (for forwarding to nurse call system 70), and the audio signals of a remotely positioned nurse that are received at communications outlet 64 (from nurse call system 70) are forwarded to a speaker onboard patient support apparatus 20.

Nurse call cable 66, in some embodiments, includes a conventional 37 pin connector on each end, one of which is adapted to be inserted into outlet 64 and the other one of which is adapted to be inserted into wall unit 60. Such 37 pin connections are one of the most common types of connectors found on existing walls of medical facilities for making connections to the nurse call system 70 and room devices 72, 74, and 78. Linked wall unit 60a and nurse call cable 66 are therefore configured to mate with one of the most common type of communication outlets 64 used in medical facilities. Such 37 pin connectors, however, are not the only type of connectors, and it will be understood that linked wall units 60a can utilize different types of connectors that are adapted to electrically couple to different types of nurse call cables 66 and/or different types of communication outlets 64. One example of such an alternative communications outlet 64 and cable 66 is disclosed in commonly assigned U.S. patent application Ser. No. 14/819,844 filed Aug. 6, 2015 by inventors Krishna Bhimavarapu et al, and entitled PATIENT SUPPORT APPARATUSES WITH WIRELESS HEADWALL COMMUNICATION, the complete disclosure of which is incorporated herein by reference. Still other types of communication outlets 64 and corresponding connectors may be utilized.

Linked wall unit 60a (FIG. 4) also includes an electrical cord 98 having a plug positioned at a far end that is adapted to be inserted into a conventional electrical outlet 100. Electrical cord 98 enables linked wall unit 60a to receive power from the mains electrical supply via outlet 100. It will be appreciated that, in some embodiments, linked wall unit 60a is battery operated and cord 98 may be omitted. In still other embodiments, linked wall unit 60a may be both battery operated and include cord 98 so that in the event of a power failure, battery power supplies power to linked wall unit 60a, and/or in the event of a battery failure, electrical power is received through outlet 100. Unlinked wall units 60b may also include a battery, electrical cord, or both.

In addition to any of the structures and functions described herein, wall units 60a (and 60b) are configured to communicate location data to patient support apparatus 20 that enables patient support apparatus 20 and/or patient support apparatus server 84 to determine the location of patient support apparatus 20 within the healthcare facility. In general, such location determination is carried out by wall units 60 sending a unique wall identifier (ID) to one or more patient support apparatuses 20 that are positioned in close proximity thereto. Patient support apparatus 20 is further adapted to determine its position relative to the wall unit 60. The combination of the patient support apparatus's relative position and the ID of the wall unit 60 is used either locally by patient support apparatus 20 to determine its position within the healthcare facility, or used remotely by server 84 to determine the position of the patient support apparatus 20 within the healthcare facility.

If determined remotely, patient support apparatus 20 may send its relative position information and/or the ID of the wall unit 60 (and its own unique patient support apparatus ID 130 (FIGS. 5 & 7) to server 84. Server 84 includes a table of all of the locations of the wall units 60 (which may be generated via a surveying operation during the installation of wall units 60), and it uses that table to correlate the patient support apparatus IDs 130 and the wall unit IDs it receives to specific locations within the healthcare facility. Thus, if a particular patient support apparatus 20 (with a particular ID 130) sends a wall unit ID that corresponds to room 430, server 84 determines that that particular patient support apparatus 20 is currently located in room 430 (and/or in a specific position relative to that wall unit 50). Server 84 determines that the particular patient support apparatus 20 is in the same room (room 430 in this example) as the wall unit 60 because each patient support apparatus 20 is configured to associate itself with a particular wall unit 60 when it is positioned within relatively close proximity thereto. Further details of this location determination process are described below, as well as in commonly assigned U.S. Pat. No. 9,999,375 issued Jun. 19, 2018, to inventors Michael Hayes et al, and entitled LOCATION DETECTION SYSTEMS AND METHODS, the complete disclosure of which is incorporated herein by reference.

In some embodiments, wall units 60 and/or patient support apparatuses 20 may be constructed to include any or all of the functionality of the wireless headwall units and/or patient support apparatuses disclosed in commonly assigned U.S. patent application Ser. No. 14/819,844 filed Aug. 6, 2015, by inventors Krishna Bhimavarapu et al, and entitled PATIENT SUPPORT APPARATUSES WITH WIRELESS HEADWALL COMMUNICATION, the complete disclosure of which is incorporated herein by reference.

In some embodiments, wall units 60 and/or patient support apparatuses 20 may also be constructed to include any or all of the functionality of the headwall units and/or patient support apparatuses disclosed in commonly assigned U.S. patent application Ser. No. 63/26,937 filed May 19, 2020, by inventors Alexander Bodurka et al, and entitled PATIENT SUPPORT APPARATUSES WITH HEADWALL COMMUNICATION, the complete disclosure of which is also incorporated herein by reference.

Still further, in some embodiments, wall units 60 and/or patient support apparatuses 20 may be constructed to include any of the features and/or functions of the headwall units 144a and/or patient support apparatuses disclosed in commonly assigned U.S. patent application Ser. No. 63/131,508 filed Dec. 29, 2020, by inventors Kirby Neihouser et al, and entitled TOOL FOR CONFIGURING HEADWALL UNITS USED FOR PATIENT SUPPORT APPARATUS COMMUNICATION, the complete disclosure of which is incorporated herein by reference.

In some embodiments, patient support apparatus 20 and/or patient support apparatus server 84 may also, or alternatively, include any or all of the functionality of the patient support apparatuses and/or patient support apparatus servers described in any of the aforementioned commonly assigned U.S. patents and/or patent applications.

FIG. 5 depicts a block diagram of patient support apparatus 20 and linked wall unit 60a. Linked wall unit 60a includes an ultra-wideband transceiver 104, a Bluetooth transceiver 106, a wall unit controller 108, configuration circuitry 110, a television controller 112, a headwall interface 114, a unit ID 116, and, in some embodiments, an infrared transceiver 118. Bluetooth transceiver 106 is adapted to communicate with a Bluetooth transceiver 122 onboard patient support apparatus 20 using RF waves in accordance with the conventional Bluetooth standard (e.g. IEEE 802.14.1 and/or the standard maintained by the Bluetooth Special Interest Group (SIG) of Kirkland, Washington, USA). In some embodiments, transceivers 106 and 122 utilize Bluetooth Low Energy communications.

Ultra-wideband transceiver 104 is adapted to communicate with one or more ultra-wideband transceivers 124 positioned onboard patient support apparatus 20. Transceiver 104 is adapted to determine a distance D1 between itself and patient support apparatus 20. Alternatively, or additionally, transceiver may be adapted to allow transceiver 124 onboard patient support apparatus 20 to determine distance D1. In some embodiments, transceivers 104 and 124 use time of flight (TOF) computations to determine distance D1. In other embodiments, transceiver 104 and 124 may utilize other techniques for determining distance D1, either in addition to, or in lieu of, TOF computations. In some embodiments, transceivers 104, 124 may also determine an angle between patient support apparatus 20 and wall unit 60 using angular information derived from antenna arrays positions onboard transceivers 104, 124, or by using other techniques.

In some embodiments, transceivers 104, 124 are implemented as any of the Trimension™ ultra-wideband modules available from NXP Semiconductors of Austin, Texas. These modules include, but are not limited to, the Trimension™ UWB modules ASMOP1BOON1, ASMOP1COOR1, and/or the ASMOP1CO0A1, that utilize any of the following chips: the NXP SR150, SR100T, SR040, NCJ29D5, and/or the OL23DO chips. Modules manufactured and/or marketed by other companies may also be used, including, but not limited to, the Decawave DWM1000, DWM10001C, DWM3000 modules (available from Decawave of Dublin, Ireland); the Nordic TSG5162 SiP module (available from Tsingoal Technology of Beijing, China); and/or the UWB hub, wand, and/or sensors available from Zebra technologies of Lincolnshire, Illinois. Still other types of UWB modules may be used to implement transceivers 104 and 124.

Wall unit controller 108 is adapted to control the operation of transceivers 104, 106, configuration circuitry 110, TV controller 112, headwall interface 114, and, if included, IR transceiver 118 (FIG. 5). When infrared transceiver 118 is included, it may be included to provide backwards compatibility to patient support apparatuses 20 that are not equipped with a UWB transceiver 124. That is, some healthcare facilities may include one or more patient support apparatuses that are not equipped with a UWB transceiver 124, but that do include an IR transceiver that is adapted to communicate with IR transceiver 118. When linked wall unit 60a includes IR transceiver 118, it is able to communicate its unit ID 116 to such patient support apparatuses via IR transceiver 118, which is a short range transceiver that is configured to only communicate with an adjacent patient support apparatus when the patient support apparatus is nearby (e.g. without about five feet or so). Such an adjacent patient support apparatus 20 then communicates the received wall unit ID 116 along with its own unique ID 130 (FIGS. 5 & 7) to server 84 which, as noted previously, is able to correlate the wall unit ID 116 to a particular location with the healthcare facility. In this manner, server 84 is able to use linked wall units 60a determine the location of versions of patient support apparatuses 20 that don't have a UWB transceiver 104, but that do have an IR transceiver.

Headwall interface 114 is adapted to change the electrical state of one or more pins that are in electrical communication with communication outlet 64 (via cable 66). Headwall interface 114 changes these electrical states in response to instructions from controller 108. For example, if exit detection system 136 of patient support apparatus 20 detects a patient exit, controller 132 sends an exit alert signal to linked wall unit 60a and controller 108 responds by instructing headwall interface 114 to change the electrical state of at least one pin that is used to signal an exit alert (or a generic priority alert) to the nurse call system 70 via communications outlet 64. In some embodiments, headwall interface 114 may be constructed in the same manner as, and/or may include any one or of the functions as, the cable interface 88 described in commonly assigned U.S. patent application Ser. No. 63/193,778 filed May 27, 2021, by inventors Krishna Bhimavarapu et al, and entitled PATIENT SUPPORT APPARATUS AND HEADWALL UNIT SYNCING, the complete disclosure of which is incorporated herein by reference. Alternatively, or additionally, headwall interface 114 may be constructed in the same manner as, and/or may include any one or more of the same functions as, the headwall interface 120 disclosed in commonly assigned U.S. patent application Ser. No. 63/131,508 filed Dec. 29, 2020, by inventors Kirby Neihouser et al, and entitled TOOL FOR CONFIGURING HEADWALL UNITS USED FOR PATIENT SUPPORT APPARATUS COMMUNICATION, the complete disclosure of which is incorporated herein by reference. Linked wall unit 60a may also be configured to perform any of the functions of the headwall units 94 disclosed in the above-mentioned '778 patent application.

Configuration circuitry 110 and TV controller 112 may be configured to perform any of the same functions as, and/or be constructed in any of the same manners as, the configuration circuitry 132 and the TV control circuit 134, respectively, of commonly assigned U.S. patent application Ser. No. 63/131,508 filed Dec. 29, 2020, by inventors Kirby Neihouser et al, and entitled TOOL FOR CONFIGURING HEADWALL UNITS USED FOR PATIENT SUPPORT APPARATUS COMMUNICATION, the complete disclosure of which has already been incorporated herein by reference. Additionally, or alternatively, linked wall unit 60a may be configured to perform any of the functions of the headwall units 144 disclosed in the aforementioned '508 patent application.

Patient support apparatus 20 includes a controller 132, a memory 134, an exit detection system 136, a microphone 138, an orientation sensor 140, and the Bluetooth transceiver 122, one or more UWB transceivers 124, and the network transceiver 94 previously mentioned (FIG. 5). Each UWB transceiver 124 is positioned at a known location on patient support apparatus 20. This known location information is stored in memory 134 and/or elsewhere, and may be defined with respect to any suitable common frame of reference. The known location information may include the spatial relationship between UWB transceivers 124 and/or any other components of patient support apparatus 20. For example, in some embodiments, the known location information includes the spatial relationship not only between UWB transceivers 124, but also the spatial relationships between UWB transceivers 124 and one or more of the following: the head end 38 of patient support apparatus 20, the foot end of patient support apparatus 20, the sides of patient support apparatus 20, the floor, and/or other components and/or landmarks of patient support apparatus 20. In some embodiments, this location information is used to determine the orientation of patient support apparatus 20 with respect to one or more walls 62, wall units 60, another patient support apparatus 20, and/or another object or structure within the healthcare facility.

Controller 132, as well as controller 108, may take on a variety of different forms. In the illustrated embodiment, each of these controllers is implemented as a conventional microcontroller. However, these controllers may be modified to use a variety of other types of circuits-either alone or in combination with one or more microcontrollers-such as, but not limited to, any one or more microprocessors, field programmable gate arrays, systems on a chip, volatile or nonvolatile memory, discrete circuitry, and/or other hardware, software, or firmware that is capable of carrying out the functions described herein, as would be known to one of ordinary skill in the art. Such components can be physically configured in any suitable manner, such as by mounting them to one or more circuit boards, or arranging them in other manners, whether combined into a single unit or distributed across multiple units. The instructions followed by controllers 108 and 132 when carrying out the functions described herein, as well as the data necessary for carrying out these functions, are stored in a corresponding memory that is accessible to that particular controller (e.g. memory 134 for controller 132, and a memory (not shown) for controller 108). In some embodiments, controller 132 may include and/or work with a microcontroller that is integrated into, or associated with, UWB transceiver(s) 124, and controller 108 may include and/or work with a microcontroller that is integrated into, or associated with, UWB transceiver 104.

Controller 132 utilizes transceiver(s) 124 to determine the relative position of patient support apparatus 20 with respect to one or more nearby wall units 60. If patient support apparatus 20 is positioned within range of a wall unit 60, its UWB transceiver 124 communicates with the UWB transceiver 104 positioned on that wall unit 60, and the two transceivers 124 and 104 exchange signals that enable them to determine the distance between themselves (i.e. distance D1 in FIG. 5). In some embodiments, UWB transceivers 104, 124 may also be configured to determine an angular relationship between themselves. The distance D1 (and angle information) in at least some embodiments is calculated by UWB transceiver 124 and controller 132 of patient support apparatus 20. In other embodiments, UWB transceiver 104 and controller 108 may calculate the distance D1 (and angle information) and forward the results of this calculation to patient support apparatus 20 (either via UWB transceiver 104 or BT transceiver 106). In either situation, patient support apparatus controller 132 is informed of the distance D1 (and, in some embodiments, as noted, the angle information) between transceiver 104 and 124. To the extent patient support apparatus 20 includes additional UWB transceivers 124, similar distance calculations are made between each of those UWB transceivers 124 and the wall unit's UWB transceiver 104.

Although FIG. 5 (and FIG. 6) only illustrate a single wall unit 60, it will be understood that a typical healthcare facility will include multiple wall units 60 positioned at different locations throughout the facility, including ones positioned within patient rooms and others positioned outside of patient rooms. Typically, at least one linked wall unit 60a will be positioned in each patient room of the healthcare facility, and if the patient room is intended to be occupied by more than one patient (e.g. it includes multiple bays), then additional linked wall units 60a may be included so that each patient support apparatus 20 will have a linked wall unit 60a positioned adjacent to each bay area in the room. Additional wall units 60, such as unlinked wall units 60b, may also be positioned at other locations through the healthcare facility.

In many locations throughout the healthcare facility, transceiver(s) 124 of an individual patient support apparatus 20 will be able to concurrently communicate with multiple wall units 60 because the communication range of the UWB transceivers 104 onboard the wall units 60 will overlap with each other. In such situations, controller 132 and/or controller 108 may utilize distances D1 between each UWB transceiver 104 and at least one of the UWB transceivers 124 positioned onboard patient support apparatus 20 to determine the location of the patient support apparatus. Such distances D1, to the extent they are not determined by controller 132 of patient support apparatus 20, are forwarded by the controllers 108 of the respective wall units 60 to patient support apparatus 20 (either via transceivers 104 or 106).

When controller 132 receives multiple distances D1 from multiple wall units 60, it may react in different manners, depending upon the particular embodiment of patient support apparatus 20 that is implemented. In a first embodiment, controller 132 forwards the multiple distances D1 to server 84 via network transceiver 94 and server 84 uses the multiple distances to determine a current location of that patient support apparatus 20 (via one or more conventional methods, such as, but not limited to, triangulation and/or trilateration). In a second embodiment, controller 132 uses the multiple distances to determine a current location of patient support apparatus 20 (via one or more of the same conventional methods) and then forwards the current location to server 84 via network transceiver 94. In another embodiment, controller 132 determines which wall unit 60 is closest to it and uses communications between only that wall unit 60 and its transceiver(s) 124 to determine its location (and thus doesn't rely on communications with multiple wall units 60 to determine its location).

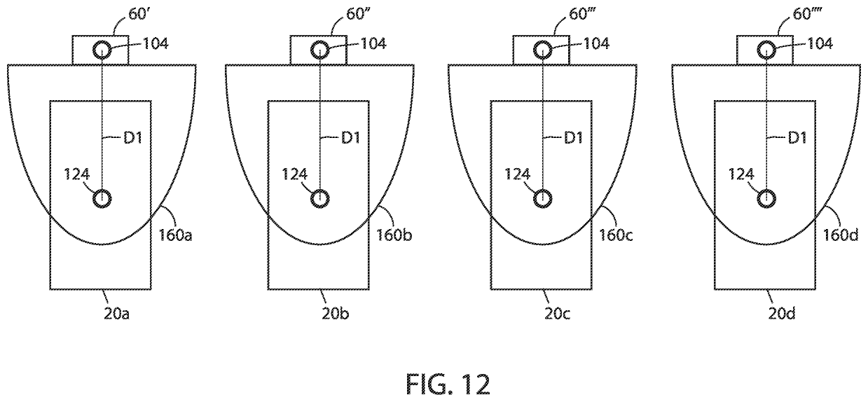

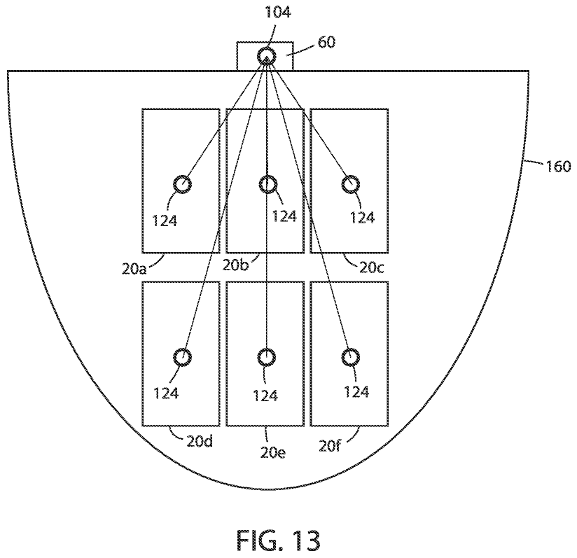

The degree of specificity of the location determined using UWB transceivers 104 and 124 may vary depending upon how close patient support apparatus 20 is to a particular wall unit 60. For example, in some embodiments, controller 132 and/or server 84 conclude that a patient support apparatus 20 is in the same location as a particular wall unit 60 if patient support apparatus 20 is within a threshold location range 160 of that wall unit 60 (discussed more below with respect to FIGS. 12 and 13). In such embodiments, the threshold location range 160 may refer to an area having horizontal dimensions of approximately five by ten feet (and a height of approximately five or more feet above the ground) that is positioned directly in front of a linked wall unit 60a, although other dimensions may, of course, be used. For unlinked wall units 60b, the threshold location range 160 may cover a greater area (and volume) of space. If the patient support apparatus 20 is positioned outside of the threshold location range 160 of a particular wall unit 60, controller 132 and/or server 84 may determine an actual coordinate location of patient support apparatus 20 and/or report a more generalized location (e.g. outside of room 402; on the third floor, in corridor X, in the Emergency Department, etc.).

When controller 132 determines multiple distances D1 (whether measured between patient support apparatus 20 and multiple wall units 60 or between patient support apparatus 20 and only a single wall unit 60), controller 132 is adapted to either use those multiple distances D1 to determine its location, or it is adapted to forward those distances to an off-board entity (e.g. server 84) to use those multiple distances to determine its location. In either case, controller 132 and/or server 84 may determine the location of patient support apparatus 20 using a coordinate frame of reference in which the position of each wall unit 60 is also known (e.g. the current position of the patient support apparatus is (X, Y, Z) in a frame of reference in which a first wall unit 60 is positioned at (X₁, Y₁, Z₁), a second wall unit is positioned at (X₂, Y₂, Z₂), a third wall unit is positioned at (X₃, Y₃, Z₃), etc.). The current position of the patient support apparatus 20 may then be compared to the one or more threshold location ranges 160 to determine if the patient support apparatus is positioned inside or outside of the threshold location ranges 160.

The distances D1 are determined by an exchange of communication signals between UWB transceivers 104 and 124. This exchange is initiated by an interrogation signal that may be sent by the UWB transceivers 104 of the wall unit 60, and/or it may be sent by the UWB transceivers 124 of the patient support apparatuses 20. The trigger for sending these interrogation signals (from either source) may simply be the passage of a predefined interval of time, in at least some embodiments. That is, in some embodiments, patient support apparatus 20 and/or wall units 60 may be configured to periodically send out an interrogation signal that will be responded to by any UWB transceivers 104 or 124 that are positioned with range of that signal. In those embodiments where patient support apparatuses 20 are configured to send out such an interrogation signal, the time intervals between the interrogation signals may be varied depending upon the location and/or other status of the patient support apparatus 20. For example, in some embodiments, patient support apparatuses 20 may be configured to send out the interrogation signals with longer timer intervals between them when the patient support apparatus is stationary, and to send out the interrogation signals with shorter time intervals between them when the patient support apparatus 20 is in motion. Indeed, in some embodiments, after patient support apparatus 20 has ceased moving, controller 132 may be configured to cease sending out such interrogation signals until it once again starts moving. In any of the aforementioned embodiments, motion of the patient support apparatus 20 may be detected in any suitable manner, such as by including one or more motion sensors on the patient support apparatus 20 (e.g. one or more accelerometers), and/or by monitoring the values of the repetitive distance measurements D1 and looking for changes indicative of movement.

The measured distances D1 (and/or angular information between wall units 60 and patient support apparatuses 20) that are generated from the communications between UWB transceivers 104, 124 may utilize Angle of Arrival (AoA) information, Time of Flight (TOF) information, Channel State Information, Time Difference of Arrival (TDoA) information, Two-Way Ranging (TWR) ranging information, and/or other information. In some embodiments, each transceiver 104, 124 includes an array of antennas that are used to generate distance and/or angular information with respect to the transceiver 104, 124 in which it is in communication. Still further, in some embodiments, transceivers 104, 124 include one or more of their own microcontrollers, and the location of transceivers 104, 124 may be determined by these internal microcontrollers without utilizing controller 132 and/or 108. In other embodiments, controllers 108 and/or 132 may work in conjunction with the microcontrollers of transceivers 104, 124 to determine their relative locations to each other.

Patient support apparatus 20 also includes, in at least some embodiments, a microphone 138 (FIG. 5) that is used to detect the voice of the patient when the patient wants to speak to a remotely positioned nurse. The patient's voice is converted to audio signals by microphone 138 and controller 132 is adapted to forward these audio signals to an adjacent communications outlet 64 positioned in wall 62 (FIG. 4). When a cable 66 is coupled between patient support apparatus 20 and outlet 64, controller 132 forwards these audio signals to outlet 64 via the cable. When no such cable 66 extends between patient support apparatus 20 and outlet 64, controller 132 wirelessly forwards these audio signals to headwall unit 60 (using transceiver 122, or in some embodiments, transceiver 124)) and controller 108 of headwall unit 60 forwards these audio signals to outlet 64. As was noted, outlet 64 is in electrical communication with a conventional nurse call system 70 that is adapted to route the audio signals to the correct nurse's station 76, and/or other location. In some embodiments, microphone 138 acts as both a microphone and a speaker. In other embodiments, a separate speaker may be included in order to communicate the voice signals received from the remotely positioned nurse. In some embodiments, the audio communication between patient support apparatus 20 and communications outlet 64 is carried out in any of the manners, and/or includes any of the structures, disclosed in commonly assigned U.S. patent application Ser. No. 16/847,753 filed Apr. 14, 2020, by inventors Alexander Bodurka et al, and entitled PATIENT SUPPORT APPARATUSES WITH NURSE CALL AUDIO MANAGEMENT, the complete disclosure of which is incorporated herein by reference.

After the installation of wall units 60a and 60b in a particular healthcare facility, the location of each wall unit 60 within that facility is recorded. In some embodiments, the coordinates of the locations of wall units 60 are recorded in a common frame of reference (or converted to a common frame of reference after recordation). Such coordinates may be three dimensional (i.e. include a height components), or they may be two dimensional (no height component). In other embodiments, a more generalized location of one or more wall units 60 is determined, rather than the precise coordinates of the wall units 60. In still other embodiments, the locations of one or more wall units 60 are determined both generally and more precisely. The generalized location of the wall units 60 may include an indication of the room, bay, area, hallway, portion of a hallway, wing, maintenance area, etc. that the wall unit 60 is positioned in. The specific location of the wall units 60, as noted, may include an X, Y, and Z coordinate within a common frame of reference.

In some embodiments, the location of each wall unit 60 (whether specific and/or general) is stored in a memory within that particular wall unit 60 and shared with the devices it communicates with (e.g. patient support apparatuses 20). In some embodiments, the location of each wall unit 60 is stored within memory 134 of each patient support apparatus 20. Still further, in some embodiments, the location of each wall unit 60 is stored within a memory accessible to server 84. Alternatively, or additionally, the location of each wall unit 60 may be stored in two or more of the aforementioned locations.

It will be appreciated that patient support apparatuses 20 are configured to communicate with wall units 60 regardless of the orientation of the patient support apparatus 20. That is, the UWB transceivers 104 and 124 are radio frequency transceivers that do not rely on line of sight communication, unlike the IR transceiver 118 (if present). Thus, the patient support apparatuses 20 do not have to be pointed in any particular direction with respect to the wall units in order for transceivers 104 and 124 to communicate. This differs from some prior art systems that use IR communication between the patient support apparatuses 20 and the wall units and that require the IR transceiver onboard the patient support apparatus to be aimed toward the wall unit in order for communication to be established.

It will also be understood that, although units 60 are referred to herein as “wall units” 60, such units do not necessarily have to be positioned on walls. That is, wall units 60 can be positioned on columns, ceilings, or any other fixed structures within the healthcare facility. It will therefore be understood that the term “wall” as used herein is not exclusive of ceilings, columns, or other fixed architectural structures.

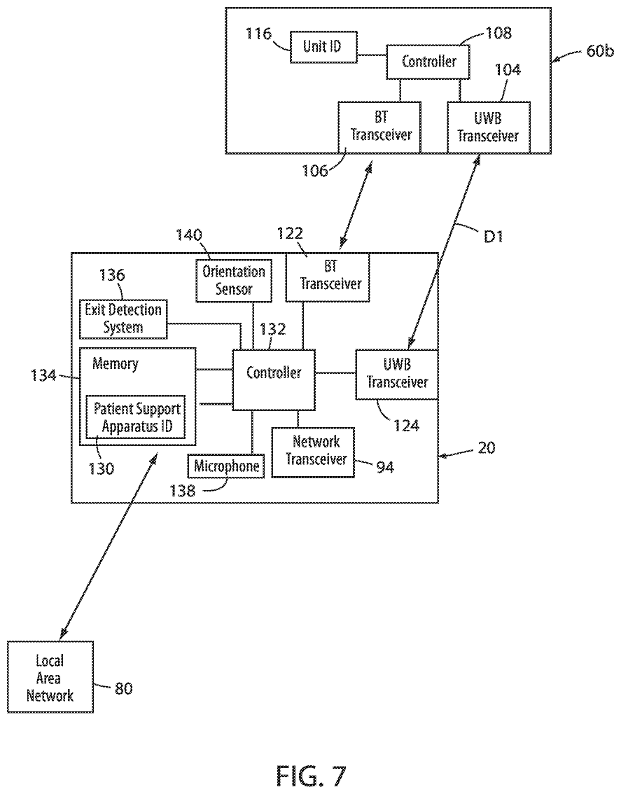

FIGS. 6 and 7 illustrate an alternative wall unit 60b that functions to provide location information to one or more patient support apparatuses 20 in the same manner as has been described herein (and as is described further below). Unlinked wall units 60b differ from linked wall units 60a in that unlinked wall units 60b are not communicatively coupled to a communications outlet 64. As a result, unlinked wall units 60b can be positioned at any fixed location within the healthcare facility, rather than only at locations that are next to a communications outlet 64. Because such communication outlets 64 are typically only placed along the headwalls within patient rooms, linked wall units 60a are typically only positioned on the headwalls of patient rooms. Unlinked wall units 60b, however, are able to be placed in hallways, in elevators, in maintenance areas, parking garages, and/or at any other desired locations where patient support apparatuses 20 are expected to travel and where location information regarding the patient support apparatuses 20 is desired.

FIG. 6 illustrates a wall unit 60b positioned in a hallway. FIG. 7 illustrates the internal components of wall unit 60b. As can be seen in FIG. 7, wall unit 60b does not include a number of components that may be found in linked wall units 60a, such as configuration circuitry 110, television controller 112, headwall interface 114, and/or IR transceiver 118. Instead, wall unit 60b includes a controller 108, Bluetooth transceiver 106, UWB transceiver 104, and a unit ID 116. These four components work in the same manner as the corresponding components of linked wall units 60a. That is, UWB transceiver 104 is used in conjunction with transceiver 124 to determine the distance D1 between wall unit 60b and a patient support apparatus 20 (and/or to determine an angular relationship between wall unit 60b and the patient support apparatus 20). Controller 108 sends unit ID 116 to patient support apparatus 20, as well as any location and/or angular calculations that it makes in response to the communications between UWB transceiver 104 and UWB transceiver 124. BT transceiver 106 may be used for any high bandwidth communications between wall unit 60b and patient support apparatus 20 that require, or are desirably carried out with, a communication channel having greater bandwidth than the communication channel between UWB transceivers 104 and 124. In some embodiments, BT transceiver 106 may be omitted entirely from unlinked wall units 60b.

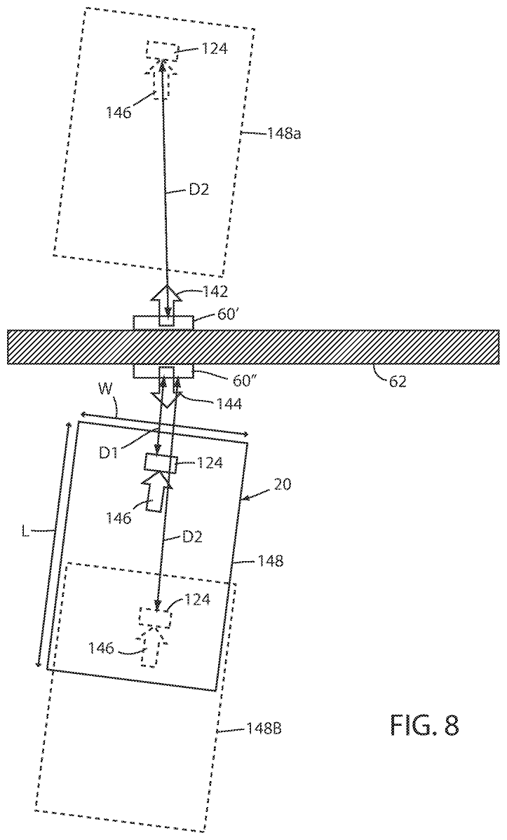

FIG. 8 illustrates one manner in which patient support apparatuses 20 may be configured to determine which wall unit 60 it is supposed to associate itself with when two or more wall units 60 are positioned relatively close to each other. Although wall units 60 are typically not placed in close proximity to each other when they are positioned on the same side of a wall 62, such close proximity may exist when wall units 60 are placed on opposite sides of a wall 62. In such situations, because UWB signals are capable of traveling through walls, unlike conventional IR signals, it is possible that a patient support apparatus 20 positioned on a first side of a wall 62 may be able to communicate with both a wall unit 60″ positioned on the same side of wall 62 as itself, as well as another wall unit 60′ positioned on the opposite side of wall 62. Still further, because of the inherent limits on the accuracy and/or precision of the distance measurements made using UWB, patient support apparatus 20 may not be able to reliably distinguish which wall unit 60 it is closer to simply based on the UWB distance measurements. Accordingly, in some aspects, patient support apparatus 20 and wall units 60 may incorporate directional information, as will now be discussed in greater detail with respect to FIG. 8.

FIG. 8 illustrates one manner in which patient support apparatus 20 may utilize its onboard orientation sensor 140 to discern whether it is located on the same side of wall 62 as a first wall unit 60′ or a second wall unit 60″. In this particular example, a first wall unit 60′ is positioned on a first side of a wall 62 that is facing away from the patient support apparatus 20, and a second wall unit 60″ is positioned on a second and opposite side of the wall 62 that is facing toward patient support apparatus 20. First wall unit 60′ is oriented in a first direction 142 and second wall unit 60″ is oriented in a second and opposite direction 144. Patient support apparatus 20 is oriented in a direction 146 that is similar to, but not necessarily the same as, first direction 142.

Patient support apparatus 20 determines its current orientation using orientation sensor 140 which, as noted, may be a magnetometer, a gyrocompass, an inertial reference unit, and/or any other sensor or combination of sensors that allows patient support apparatus 20 to determine it geographical orientation (e.g. whether it is pointing north, south, east, west, etc.). Wall units 60′ and 60″ may also include one or more orientation sensors that enable their respective controllers 108 to determine the orientation of themselves. Alternatively, during installation of wall units 60′ and 60″, the orientation of wall units 60′ and 60″ may be determined by an external sensor used by a surveying technician, and this orientation may then be transferred to a memory within the wall units 60′, 60″, a memory inside of patient support apparatus 20 (e.g. memory 134), and/or a memory inside of (or accessible to) server 84. In either situation, the orientation of wall units 60′ and 60″ is able to be determined, either from reading an onboard orientation sensor, or from reading the orientation from a memory in which it is stored. To the extent wall units 60′ and 60″ contain an orientation sensor or have their orientations stored in an internal memory, controller 108 forwards this orientation to patient support apparatus 20, using either Bluetooth transceiver 106 and/or UWB transceiver 104. This orientation information is forwarded to the patient support apparatus 20 along with the unique unit IDs 116 of each wall unit 60′ and 60.″

After receiving the orientations and unit IDs of wall units 60′ and 60″, controller 132 compares those orientations to the current orientation of patient support apparatus 20 and determines which, if any, of wall units 60′ or 60″ are facing toward patient support apparatus 20. In the example shown in FIG. 8, wall unit 60″ is facing in second direction 144, which is substantially opposite direction 146 of patient support apparatus 20, and second wall unit 60″ is therefore facing toward patient support apparatus 20. In contrast, first wall unit 60′, which is on the other side of wall 62, is facing substantially in the same direction 146 as patient support apparatus 20. First wall unit 60′ and patient support apparatus 20 are therefore facing in the same direction 142, not toward each other.

It will be understood that it is not necessary for the direction 146 of the patient support apparatus 20 to be exactly 180 degrees opposite to that of one of the directions 142, 144 of the wall units 60′ and 60″ in order for controller 132 to determine which side of wall 62 it is currently positioned on. Generally speaking, controller 132 is able to discern which side of wall 62 is it positioned on by determining whether the direction 146 of patient support apparatus 20 is more aligned with the direction 142 of wall unit 60′ or more aligned with the direction 144 of wall unit 60″. Whichever direction it is more aligned with (i.e. whichever direction it is closer to), that is the direction of the wall unit 60 that is positioned on the opposite side of wall 62. In the example of FIG. 62, it can therefore be seen that patient support apparatus 20 is pointed in a direction 146 that is nearly the same as direction 142, and therefore wall unit 60′ is positioned on the opposite side of wall 62. In those situations where direction 146 of patient support apparatus 20 is exactly 90 degrees different from both of directions 142 and 144, it may be useful to include a second and/or third UWB transceiver 146 on patient support apparatus 20 and utilize information from those additional sensors 146 to determine which side of wall 62 patient support apparatus 20 is positioned on, as will be discussed in greater detail below. Alternatively, or additionally, angular information may be derived from the communication between the transceivers 104 of wall units 60′a and 60″ and the transceivers 124 of patient support apparatus 20, and this angular information may be used to resolve the question of which wall unit 60′ or 60″ is positioned on the same side of wall 62 as patient support apparatus 20.

As was noted above, the orientation information used in the example shown in FIG. 8 is useful because of the fact that UWB signals are often able to pass through the walls of a healthcare facility. As a result, if two wall units 60 are positioned relatively close to each other, but on opposite sides of a wall, patient support apparatus 20 may end up being positioned an absolute distance from each wall unit that is substantially similar (i.e. patient support apparatus 20 may end up being positioned relatively close to both wall units 60, albeit with one of the wall units 60 positioned on the other side of the wall 62). Because the distances measured by UWB transceiver 124 between itself and each of the wall units 60′ and 60″ may be similar, and because the accuracy of these distance measurements has an inherent uncertainty, controller 132 may not be able to determine which wall unit 60′ or 60″ it is closer to based solely on the distance measurements to each wall unit.

In the example of FIG. 8, controller 132 may be further configured to utilize known dimensions of patient support apparatus 20 to determine which side of a wall 62 it is currently positioned on (including situations where another wall unit (e.g. wall unit 60′) may be not present). Such known dimensions may include, but are not limited to, a length L of patient support apparatus 20 and a width W of patient support apparatus 20. Thus, in some aspects, memory 134 contains length L and width W stored therein and these dimensions are accessible to controller 132. In the particular example shown in FIG. 8, controller 132 may utilize either or both of these dimensions to determine that it is positioned at the location 148 shown in FIG. 8, rather than at potential location 148a. When patient support apparatus 20 is positioned at the location 148 shown in FIG. 8, it is able to conclude that it is not positioned at potential location 148a by comparing a measurement of distance D1 with length L of patient support apparatus 20. That is, because distance D1 is less than the length L of patient support apparatus 20, controller 132 is able to conclude that patient support apparatus 20 is positioned at location 148, rather than potential location 148a. This is because patient support apparatus 20 cannot position itself a distance D1 away from a wall unit 60 while facing away from the wall unit 60 (such as in potential position 148a) because, when facing away from the wall unit, the signals from the UWB transceiver 124 have to travel a distance to the wall unit, due to the length L of patient support apparatus, that is greater than distance D1.

In some situations, however, patient support apparatus 20 may be positioned at a location where it is not possible for controller 132 to resolve which side of wall 62 it is positioned on utilizing only distance measurements and the relative directions 142, 144, and/or 146. For example, controller 132 may not be able to differentiate between potential position 148a and 148b without additional information. As shown in FIG. 8, potential position 148b is located the same distance D2 from wall unit 60″ as the distance D2 between potential location 148a and wall unit 60′. Further, patient support apparatus 20 is oriented in the same direction 146 in both potential positions 148a and 148b. Unless controller 132 is able to communicate with other wall units 60 (not shown in FIG. 8) and use that communication to resolve its location with respect to wall 62, controller 132 may not be able to distinguish between positions 148a and 148b if it only utilizes distance measurements. Thus, it may be desirable, in some aspects, to also use UWB transceivers 104 and 124 to determine their angular relationship, thereby enabling controller 1323 to determine which side of the wall 62 the wall unit 60′ and/or 60″ is positioned on. Alternatively, it may be desirable to include more than one UWB transceiver 124 on patient support apparatus 20 in order for controller 132 to be able to differentiate between different positions, such as positions 148a and 148b.

FIG. 9 illustrates an example of a patient support apparatus 20 that includes at least three UWB transceivers 124a-c. The use of multiple transceivers 124a-c allows controller 132 to distinguish between potential positions, such as potential positions 148a and 148b. In the particular example of FIG. 8, controller 132 is able to distinguish between potential positions 148a and 148b because the distances between the wall unit 60′ and/or 60″ and the UWB transceivers 124b and 124c will be different for positions 148b and 148c. This is explained in greater detail with respect to FIG. 9 and potential positions 148c and 148d.

In the example of FIG. 9, a patient support apparatus 20 that is located in position 148c will make measurements D1, D2, and D3 between wall units 60″ and UWB transceivers 124a, 124b, and 124c, respectively. A patient support apparatus 20 that is positioned in position 148d will make measurements D4, D5, and D6 between wall unit 60′ and UWB transceivers 124a, 124b, and 124c, respectively. Controller 132 is able to distinguish between positions 148c and 148d because, when in position 148d, the distance D5 of transceiver 124b is greater than the distance D6 of transceiver 124c. However, when patient support apparatus 20 is in position 148c, the distance D2 of transceiver 124b is shorter than the distance D3 of transceiver 124c. Thus, by knowing the relative locations of each of transceivers 124a-c on patient support apparatus 20, as well as the distance measurements to each of them from the wall unit (60′ and/or 60″), controller 132 is able to determine which side of the wall 62 is it positioned on. In the example of FIG. 9, controller 132 determines that wall unit 60″ is positioned on the same side of wall 62 as position 148c because the distance measurement of transceiver 124c is greater than that of transceiver 124b, which is the opposite of what it would be were patient support apparatus 20 positioned in potential location 148d. This position determination may be made in combination with a comparison of the direction 146 of patient support apparatus 20 relative to the directions 142 and 144 of wall units 60′ and 60″, respectively.

It will be understood that, although FIGS. 8 and 9 illustrate two wall units 60′ and 60″ that are positioned adjacent to each other on opposite sides of a wall 62, controller 132 of patient support apparatus 20 may use the same logic described above to determine what side of a wall 62 it is positioned one when there is only one wall unit 60 that is present. In other words, if wall unit 60′ were removed from the examples shown in FIGS. 8 and 9, controller 132 uses the same logic to determine whether it is on the same side of wall 62 as wall unit 60″ or not. The logic is therefore applicable to situations when only a single wall unit is present, as well as when multiple wall units 60 are within communication range.

It will also be understood that, although FIGS. 8 and 9 illustrate two wall units 60′ and 60″ that are positioned right next to each other along wall 62 (albeit on opposite sides), controller 132 uses the same logic described above when one of wall units 60′ or 60″ is displaced with respect to the other one along the length of the wall 62. In other words, if, for example, wall unit 60′ were shifted to the right in FIG. 8 or 9 (and wall unit 60″ remained in the position illustrated), controller 132 is configured to utilize the same logic to determine whether it is positioned on the same or opposite side of wall 62 as wall unit 60′, and/or whether it is positioned on the same or opposite side of wall 62 as wall unit 60″.

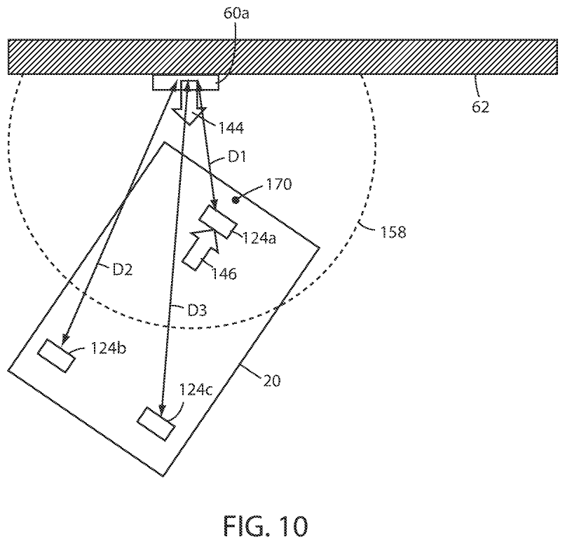

FIG. 10 illustrates an example of a patient support apparatus 20 positioned next to a linked wall unit 60a wherein the controller 132 onboard patient support apparatus 20 determines whether or not to associate with the linked wall unit 60a. In some aspects, each linked wall unit 60a includes an association threshold 158, which defines a range (or a volume of space) within which a patient support apparatus 20 must be positioned before the patient support apparatus 20 associates itself with that particular linked wall unit 60a. Although FIG. 10 depicts association threshold 158 as being generally circularly shaped with its center being aligned with the approximate center of linked wall unit 60a, it will be understood that the shape of association threshold 158 can be changed to other types of shapes. In some aspects, each wall unit 60a includes within its memory a definition of the association threshold 158, which may include both the shape and the distance(s) of threshold 158 from that particular linked wall unit 60a. In such aspects, controller 108 of the linked wall unit 60a may share the definition of its association threshold 158 with patient support apparatus 20 when the patient support apparatus 20 is positioned within communication range of that particular linked wall unit 60a.

Controller 132 of patient support apparatus 20 is configured to associate itself with a particular linked wall unit 60a when a defined reference point 170 (or points 170) on patient support apparatus 20 is positioned at a location that falls within association threshold 158. The reference point 170 (or points 170) is stored in memory 134, and its location (or their locations) is stored such that the relative positions of each of transceivers 124a-c is known with respect to the reference point 170 (or reference points 170). Controller 132 uses the distance measurements D1-D3 (FIG. 10), along with the known location of reference point 170 (and, in some instances, the output of orientation sensor 140) to determine whether reference point 170 (or reference points 170, if there are multiple) falls within association threshold 158. As can be seen in the example shown in FIG. 10, reference point 170 is within threshold 158, and controller 132 is therefore configured to automatically associate itself with wall unit 60a for as long as reference point 170 remains within threshold 158.

The term “associates,” or its variants, as used herein, refers to the identification by controller 132 of the linked wall unit 60a that controller 132 will send data to for forwarding to the associated communication outlet 64, as well as the linked wall unit 60a that controller 132 will receive data from that originated from communication outlet 64. The associated wall unit 60a is therefore the wall unit 60a that patient support apparatus 20 will send the patient's voice signals to (and/or exit detection alerts to) for forwarding to nurse call system 70. It is also the wall unit 60a that controller 132 will send television commands to when a patient onboard patient support apparatus 20 activates one or more of the television controls 50l-50r. Similarly, it is the wall unit 60a that controller 132 will send light commands to when a patient onboard patient support apparatus 20 activates one or more or the reading or room light controls 50s or 50t. The linked wall unit 60a that patient support apparatus 20 associates itself with is also the wall unit 60a that patient support apparatus 20 will receive audio signals from and direct to its onboard speaker(s). Such audio signals may correspond to voice signals from a remotely positioned nurse that are forwarded to the corresponding communication outlet 64 by way of nurse call system 70, or such audio signals may correspond to television audio signals that are routed from television 72 to communication outlet 64 by way of the one or more conductors 68.

Thus, in the example shown in FIG. 10, when reference point 170 is positioned inside of association threshold 158, controller 132 is configured to automatically associate itself with the linked wall unit 60a shown therein and exchange audio signals for forwarding to/from the communication outlet 64 (not shown in FIG. 10) that linked wall unit 60a is coupled to by way of cable 66 (also not shown). Once associated with linked wall unit 60a, controller 132 is further adapted to forward television commands to linked wall unit 60a for forwarding to television 72. The association thresholds 158 of each wall unit 60a are defined such that they do not overlap with the association thresholds 158 of any other wall units 60a. Thus, a patient support apparatus 20 can only be associated with a single wall unit 60a at any given time.

It will be understood that, in some embodiments, unlinked wall units 60b do not have an association threshold 158 associated with them. This is because, as noted previously, unlinked wall units 60b are not coupled to a corresponding communication outlet 64, and therefore can neither forward signals from patient support apparatus 20 to another entity (e.g. nurse call system 70 and/or television 72), nor can they receive signals from another entity (e.g. nurse call system 70 and/or television 72) and forward them to patient support apparatus 20. Accordingly, when a patient support apparatus 20 is positioned close to an unlinked wall unit 60b, it is configured to determine is relative location to that unlinked wall unit 60b, but it does not forward any audio signals to, nor receive any audio signals from, the unlinked wall unit 60b.

It will be understood that, in an alternative embodiment, any of the distance measurements, wall-side determinations, and/or association determinations discussed above may be performed, either wholly or partially, by patient support apparatus server 84 instead of by controller 132 and/or controller 108. Thus, for example, in some embodiments, patient support apparatus 20 reports the distance measurements it makes with respect to one or more wall units 60 (e.g. distance D1 in FIG. 8), the corresponding unit IDs 116, its ID 130, its orientation, and, in some situations, the orientations of wall units 60′ and 60″ to server 84. Server 84 then uses this information to determine which wall unit 60′ or 60″ patient support apparatus 20 is facing, and therefore what location patient support apparatus 20 is currently in. In other words, in some embodiments, the calculation of the location of patient support apparatuses 20 and/or their associations is off-loaded from the patient support apparatuses themselves to server 84, while in other embodiments, this calculation is performed, either wholly or partially, onboard patient support apparatuses 20 by controller 132 and then reported to server 84.

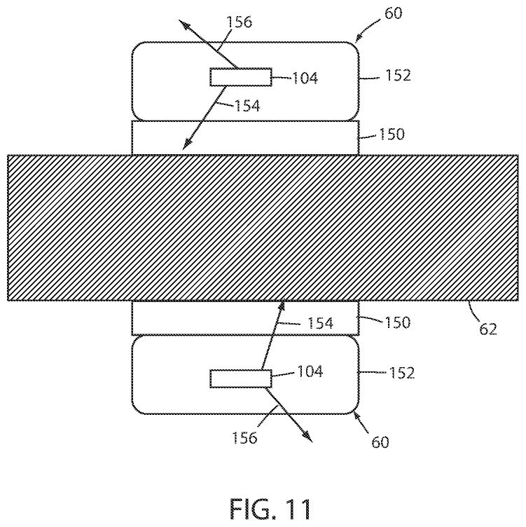

FIG. 11 illustrates a modified embodiment of wall units 60 that may be utilized with any of the patient support apparatuses 20 disclosed herein. In this modified embodiment, wall units 60 include a shielding layer 150 that is coupled to a rear side of a main housing 152 such that when the wall unit 60 is attached to a wall 62, the shielding layer 150 is positioned between the main housing 152 and the wall 62. The shielding layer is adapted to attenuate those UWB signals emitted by the UWB transceiver 104 that are directed toward wall 62 without effecting the UWB signals that are emitted away from wall 62. Thus, when a UWB transceiver 104 within a wall unit 60 emits a UWB signal in a direction 154 toward its nearest wall 62, those signals are attenuated by the shielding layer 150 such that, to the extent those signals are able to travel through wall 62, a patient support apparatus 20 positioned on the opposite side of the wall 62 will not detect them because they are too weak, and/or patient support apparatus 20 will be easily able to distinguish those signals from ones emitted by another wall unit 60 that is positioned on the same side of wall unit 60 as patient support apparatus 20 is. On the other hand, signals emitted by transceiver 104 that are emitted outwardly and away from the wall 62, i.e. in a direction 156, do not travel through shielding layer 150, and are therefore not attenuated by shielding layer 150. As a result, those signals from UWB transceiver 104 that are directed into the room or hallway in which wall unit 60 is positioned will be strongest, while those signals that may travel through the wall positioned behind the wall unit 60 will be severely attenuated, thereby limiting the ability of patient support apparatuses 20 to detect wall units 60 that are positioned on opposite sides of the walls within a healthcare facility.

Shielding layer 150 may be comprised of any conventional metallic material or other material that is known to attenuate radio frequency signals. In some embodiments, shielding layer may be made out of, or include, a mu metal, which is a metal with a high magnetic permeability (u). In some embodiments, shielding layer 150 may include multiple sub-layers, and one of such sub-layers may be made of mu metal, or another shielding layer. Shielding layer 150 is attached to a rear side of main housing 152 so that when wall unit 60 is attached to a wall 62, shielding layer 150 is positioned between main housing 152 and the wall 62. Main housing 152 includes all of the components shown in wall units 60a and 60b of FIGS. 5 and 7, respectively. That is, main housing 152 includes the electronics and other components of wall units 60, while shielding layer may only include metal or other materials that attenuate the UWB signals.

It will be understood that shielding layer 150 may be added to any of the embodiments of wall units 60 discussed herein. Thus, in some embodiments, the wall units 60 of FIG. 8 may be modified to include shielding layer 150, in which case the patient support apparatuses 20 interact with wall units 60 whose orientations are known or determinable (as discussed above with respect to FIG. 8). In such embodiments, the combination of the orientation of wall units 60 and the shielding of the UWB signals from those wall units 60 that are positioned on the opposite side of a wall 62 enable patient support apparatus 20 to more easily and more accurately determine which wall unit 60 is on the same side of the wall 62 as patient support apparatus 20. Alternatively, shielding layer 150 may be omitted from the wall units of FIG. 8 and/or from any of the other wall units 60 discussed herein. If shielding layer 150 is included, it may be included with both wall units 60a and 60b, or it may be included in only one of these two types of wall units.

FIG. 12 illustrates an arbitrary arrangement of a plurality of patient support apparatuses 20a-d whose locations are individually associated with a distinct wall unit 60 in a one-to-one fashion. That is, each patient support apparatus 20 has its own wall unit 60 positioned nearby that it informs the patient support apparatus of its current location. Thus, as shown in FIG. 12, a first patient support apparatus 20a has associated itself with a first wall unit 60′, a second patient support apparatus 20b has associated itself with a second wall unit 60″, a third patient support apparatus 20c has associated itself with a third wall unit 60″, and a fourth patient support apparatus 20d has associated itself with a fourth wall unit 60″.

Each wall unit 60 has associated with it a threshold location range 160 (or threshold location distance(s)). The threshold location range 160 defines a distance (and in some cases orientation information and/or multiple distances) within which patient support apparatus 20 (or a reference point on patient support apparatus, such as reference point 170) must be located in order for the patient support apparatus 20 to be considered to be located at the same location as the wall unit 60. Thus, for example, because UWB transceiver 124 of patient support apparatus 20a is positioned within the threshold location range 160 of wall unit 60′, controller 132 concludes that patient support apparatus 20 is positioned at the same location as wall unit 60′ and therefore reports its location to server 84 as being the same as wall unit 60′. This reporting of its location may be made with respect to a particular room, bay area, or other designated area. Thus, patient support apparatus 20a may report to server 84 that it is currently at bed bay B of room 444 because transceiver 124 is currently positioned inside of threshold location range 160a. On the other hand, if transceiver 124 of patient support apparatus 20a were positioned outside of threshold location range 160a, patient support apparatus 20 might report to server 84 less specific location, such as it being currently in room 444 (but not in a particular bay), or on a particular floor (but with no room number and/or bay number), or in a particular hallway (but with no specific location within that hallway), or it might report the value of D1 along with the wall unit ID 116 of wall unit 60′.

It can therefore be seen that, in at least some embodiments, patient support apparatuses 20 are configured to report different degrees of location specificity, depending upon whether or not they are within the corresponding threshold location distance 160 of a nearby wall unit 60. In some embodiments, the corresponding threshold location distances 160 may vary from wall unit 60 to wall unit 60. For example, a wall unit 60 that is positioned within a large maintenance and/or storage area where it is not necessary to know the precise position of a patient support apparatus 20 might have a larger threshold location distance 160 than a wall unit 60 that is placed adjacent a bay area of a patient room. The threshold location range 160 of a particular wall unit 60 may be stored within that particular wall unit 60 and transmitted to nearby patient support apparatuses 20, or patient support apparatuses 20 may store in memory 134 all of the threshold location ranges 160 for all of the wall units 60, or server 84 may store these threshold location ranges 160 for each wall unit 60 (or combination of these configurations may be used).

It should be understood that the threshold location distances 160 are not the same as the range of UWB transceivers 104, but instead are defined distances that are less than the maximum communication range of transceivers 104. Therefore, it is still possible for a patient support apparatus 20 to communicate with a transceiver 104 of a wall unit 60 when that patient support apparatus 20 is positioned outside of the threshold range 160. Being outside of the range 160 merely determines what location information the patient support apparatus 20 will report to server 84.

It should also be understood that the threshold location distances 160 may be separate and independent from the association thresholds 158 discussed previously (see FIG. 10). Threshold location distances 160 refer to distances within which a patient support apparatus 20 must be located in order for the location of that patient support apparatus 20 to be considered the same as that of the corresponding wall unit 60. Association range 158 refers to the range within which a patient support apparatus 20 must be located in order for the patient support apparatus 20 to associate itself with that particular wall unit 60 (and to thereafter carry out the communications discussed above with the wall unit that utilize the associated communication outlet 64). In at least one embodiment, each linked wall unit 60a has a threshold location distance 160 and an association threshold 158 that may or may not be the same, and each unlinked wall unit 60b has only a threshold location distance 160 and no association threshold 158. Other combinations of thresholds 158 and 160 may be implemented with respect to wall units 60a and/or 60b.

As was noted in above, FIG. 12 illustrates four patient support apparatuses 20a-d that are each positioned within the threshold range 160a-d of a corresponding wall unit 60′-60″. For some locations of a healthcare facility, it may be desirable to have a threshold range 160 that is large enough to allow multiple patient support apparatuses 20 to concurrently be positioned within that threshold range 160. An example of such a situation is shown in FIG. 13. FIG. 13 shows a single wall unit 60 having a relatively large threshold range 160 that might be useful for a storage area, maintenance area, or other area of a healthcare facility where a large number of patient support apparatuses 20 might be positioned and where the precise location of those patient support apparatuses 20 within the threshold range 160 might be unnecessary.

In the example shown in FIG. 13, UWB transceiver 104 of wall unit 60 is able to concurrently communicate with six patient support apparatuses 20 that are positioned within threshold location range 160. Each patient support apparatus 20a-f that is positioned within threshold range 160 may report its location to server 84 as simply corresponding to the room, hallway, or other area that corresponds to the location of the wall unit 60. Therefore, for example, if wall unit 60 is positioned in storage room B (or wall unit 60 is given the designation of “storage room B”) all of the patient support apparatuses 20a-f may simply report their current location to server 84 as being in storage room B. Further granularity as to the position of the patient support apparatuses 20 within storage room B (e.g. their distances from wall unit 60) may be omitted from the location information sent to server 84.

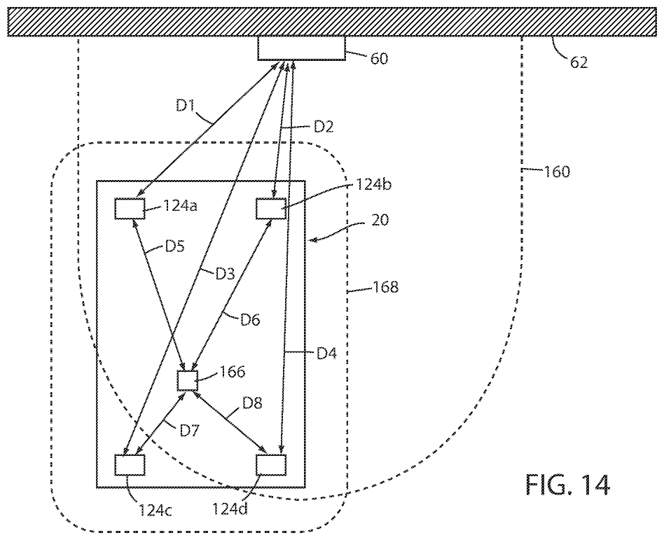

FIG. 14 illustrates a patient support apparatus 20 that includes four UWB transceivers 124a-d. Patient support apparatus 20 is configured to use at least one, if not more than one, of UWB transceivers 124a-d to determine their respective distance D1 (or D2, D3, or D4) from a nearby wall unit 60 in the manner previously discussed. In some embodiments, patient support apparatus 20 uses the distance measurements D1-D4, along with the known position of each transceiver 124 relative to each other, to determine the orientation of patient support apparatus 20 with respect to wall unit 60. Still further, in some embodiments, patient support apparatus 20 may be configured to use transceivers 124 to determine a location of a tagged medical device 166 that is positioned within the vicinity of patient support apparatus 20.

More specifically, patient support apparatus 20 may be configured to determine if a tagged medical device 166 is positioned within a predetermined volume of space 168 that is defined around patient support apparatus 20. The results of this location determination are forwarded to patient support apparatus server 84 via network transceiver 94 and server 84 is configured to share the results with one or more software applications that may be executed by any one or more computer devices that are in communication with server 84 (e.g. one or more electronic devices 96). Patient support apparatus 20 and/or server 84 may be configured to automatically associate a medical device 166 with a particular patient support apparatus 20 if the medical device is positioned within the volume of space 168, and to not associate the medical device 166 with the particular patient support apparatus 20 if the medical device 166 is positioned outside of the volume of space 168.

When patient support apparatus 20 and/or server 84 associate a medical device 166 with a particular patient support apparatus 20, controller 132 and/or server 84 are configured to inform medical personnel (via electronic devices 96) that the medical device 166 is associated with a particular patient support apparatus 20 and/or with a particular patient assigned to that patient support apparatus 20. In this manner, data from the medical device 166 can be correlated with a particular patient. Indeed, in some embodiments, patient support apparatus 20 is configured to automatically establish communication with the medical device 166 (via transceiver 124 and/or Bluetooth transceiver 122) and to forward data from that device to server 84, which in turn may automatically forward the data to the corresponding patient's electronic medical record. Because patient support apparatus 20 automatically determines that the medical device 166 is positioned within space volume 168, it is not necessary for a caregiver to take any manual steps to ensure that data from medical device 166 is forwarded to the proper corresponding patient's electronic medical record because patient support apparatus 20, along with server 84, automatically determine the correct patient associated with that medical device. Further details regarding at least one manner in which this automatic patient determination may be made are found in commonly assigned U.S. patent application Ser. No. 63/193,777 filed May 27, 2021, by inventors Thomas Deeds et al, and entitled SYSTEM FOR ASSOCIATING MEDICAL DEVICE DATA, the complete disclosure of which is incorporated herein by reference.

In addition, the aforementioned '777 patent discloses several manners in which the tag coupled to medical device 168 may be constructed, as well as the communications that take place in order for the position of the tag to be automatically determined. These disclosures, as well as the entire disclosure of the '777 patent application, are incorporated herein in their entirety by reference, and any one or more of the functions of the anchors and pseudo-anchors disclosed therein may be incorporated herein into any one or more of the UWB transceivers disclosed herein.

In general, the tag attached to a medical device 166 includes a UWB transceiver that is able to communicate with each UWB transceiver 124. This communication enables controller 132 of patient support apparatus 20 to determine the distances D5-D8 (FIG. 14) between each UWB transceiver 124 and the tag attached to, or built into, the medical device 166. By knowing distances D5-D8, as well as the location of each transceiver 124 on patient support apparatus 20, controller 132 is able to determine the relative position of the tagged medical device 166 with respect to patient support apparatus 20, including whether the tagged medical device 166 is positioned inside or outside of space volume 168.

It will be understood that a tag may be attached to any suitable medical device 166 and have its location determined with respect to patient support apparatus 20 and space volume 168. Such medical devices include, but are not limited to, exercise devices, heel care boots, IV stands and/or poles, infusion pumps, DVT pumps, ventilators, patient sensors (e.g. saturated oxygen (SpO₂) sensors, EKG sensors, vital sign sensor, etc.), patient positioning devices (e.g. wedges, turning devices, pumps), ambient sensors (e.g. air temperature, air flow, light, humidity, pressure, altitude, sound/noise), mattress 42, an incontinence pad or one or more sensors adapted to detect patient incontinence, a Holter device adapted to monitor and record a patient's heart signals, a patient ID tag or bracelet worn by the patient that identifies the patient, a caregiver tag or ID bracelet worn by a caregiver that identifies the caregiver, one or more pieces of furniture that a patient may be expected to use, and/or other types of devices. In general, medical devices 62 may include any devices that are used in a medical setting for treating, diagnosing, monitoring, and/or caring for a patient.

In at least one embodiment, patient support apparatus 20 of FIG. 14 may be configured to detect the position of an exercise device (and/or movement of the exercise device) in any one or more of the manners disclosed in commonly assigned U.S. patent application Ser. No. 63/161,175 filed Mar. 15, 2021, by inventors Krishna Bhimavarapu et al, and entitled EXERCISE DEVICE AND PATIENT SUPPORT APPARATUS, the complete disclosure of which is incorporated herein by reference.

In some embodiments, the transceivers 104, 124 may operate in the same manner as, and include any of the same functions as, the anchors and pseudo-anchors disclosed in commonly assigned U.S. patent application Ser. No. 63/193,777 filed May 27, 2021, by inventors Thomas Deeds et al, and entitled SYSTEM FOR ASSOCIATING MEDICAL DEVICE DATA, the complete disclosure of which has already been incorporated herein by reference.

In some embodiments, wall units 60 may also be utilized to determine the location of a medical device 166, such as is disclosed in commonly assigned U.S. patent application Ser. No. 63/132,514 filed Dec. 31, 2020, by inventors Alexander Bodurka et al, and entitled PATIENT SUPPORT APPARATUS AND MEDICAL DEVICE NETWORKS, and in commonly assigned U.S. patent application Ser. No. 63/154,677 filed Feb. 27, 2021, by inventors Celso Pereira et al, and entitled SYSTEM FOR DETERMINING PATIENT SUPPORT APPARATUS AND MEDICAL DEVICE LOCATION, the complete disclosures of both of which are incorporated herein by reference.

In addition to determining the location of a medical device 166 positioned within the vicinity of patient support apparatus 20, patient support apparatus 20 may further be configured to determine the type (and/or other identity) of the medical device 166. The tag attached to the medical device 166 and/or the medical device 166 itself may include a device ID that identifies the type of medical device that the medical device 166 is (e.g. a heel care boot, a vital signs monitor, a patient monitor, an IV stand, a therapy device, etc.). The device ID may also or alternatively include an ID that uniquely identifies medical device 166 such that it can be distinguished from other medical devices 166 of the same type. The medical device ID may be transmitted from medical device 166 to one or more of the transceivers 124 onboard patient support apparatus 20, and patient support apparatus 20 is configured to then forward the ID to server 84 and/or one or more electronic devices 96. The recipient of the medical device ID has access to a data table that correlates the ID to a specific type of medical device, and the recipient can then share the fact that a particular type of medical device 166 is being used with a patient on a particular patient support apparatus 20. This sharing may take place via one or more of the electronic devices 96, thereby enabling the electronic devices 96 to display the type of medical device being used with a particular patient. Data from the medical device 166 may also be displayed on the same electronic device 96, thereby giving the viewer real time information about the medical devices 166 being used with a particular patient support apparatus.

In any of the embodiments disclosed herein, server 84 may be configured to additionally execute a caregiver assistance software application of the type described in the following commonly assigned patent applications: U.S. patent application Ser. No. 62/826,097, filed Mar. 29, 2019 by inventors Thomas Durlach et al, and entitled PATIENT CARE SYSTEM; U.S. patent application Ser. No. 16/832,760 filed Mar. 27, 2020, by inventors Thomas Durlach et al, and entitled PATIENT CARE SYSTEM; and/or PCT patent application serial number PCT/US2020/039587 filed Jun. 25, 2020, by inventors Thomas Durlach et al, and entitled CAREGIVER ASSISTANCE SYSTEM, the complete disclosures of which are all incorporated herein by reference. That is, server 84 may be configured to share with one or more electronic devices any of the information shared with the electronic devices disclosed in these aforementioned patent applications. Thus, for example, server 84 may be configured to not only share the location of patient support apparatuses 20 (and any medical devices that may be associated with them) with electronic devices 96, but it may also forward patient support apparatus status data (e.g. current siderail position, bed exit status, brake status, height status, scale data, etc.) and/or caregiver rounding information (e.g. when the last rounding was performed for a particular patient, when the next rounds are due, etc.).

In some embodiments, wall units 60a and/or 60b may be configured to send a signal to patient support apparatus 20 (via UWB transceiver 104 and/or via BT transceiver 106) indicating what type of wall unit it is (e.g. whether it is a linked wall unit 60a or an unlinked wall unit 60b). Controller 132 of patient support apparatus 20 uses this information to determine whether or not to forward audio signals from microphone 138 to the wall unit 60 via BT transceiver 122 or UWB transceiver 124. More specifically, if the wall unit is a linked wall unit 60a, controller 132 forwards the audio signals (and linked wall unit 60a then forwards the audio signals to the communications outlet 64). If the wall unit is an unlinked wall unit 60b, controller 132 does not forward the audio signals (but instead forwards them to a nurse call cable port that receives a nurse call cable 66). The audio signals are generated from the patient's voice when he or she is talking to a remotely positioned caregiver via the healthcare facility's nurse call system.

Patient support apparatus 20 may be configured in some embodiments to communicate with additional types of wall units 60 beyond the specific wall units 60a and 60b described herein. For example, some healthcare facilities may include IR-based wall units that do not have any UWB transceivers 104 included within them, but instead have just the BT transceiver 106 and an IR transceiver 118. Patient support apparatuses 20 may be configured to include an IR transceiver in addition to the BT and UWB transceivers 122 and 124 that enables them to communicate with the IR transceiver 118 included in such IR-based wall units. In such embodiments, patient support apparatus 20 determines its location with respect to the IR-based wall unit by successfully establishing communication between its IR transceiver and IR transceiver 118. When such communication is successfully established (which only happens when patient support apparatus 20 is positioned within about five to ten feet of the IR-based wall unit), the IR-based wall unit shares its unit ID 116 with the patient support apparatus 20 and controller 132 forwards this unit ID 116 to server 84 (which may then correlate the unit ID 116 to a particular location). Patient support apparatuses 20 may therefore be configured to determine their location from wall units 60 in two different manners, depending upon the wall unit type: (1) via UWB communication and distance determinations, or (2) or via short range IR communication that is line-of-sight and only possible when positioned within close proximity to an IR-based wall unit. The IR-based wall unit, in some embodiments, includes all of the same components of linked wall unit 60a shown in FIG. 5 with the sole exception of UWB transceiver 104. The IR communication between patient support apparatus 20 and the IR-based wall unit may be triggered by patient support apparatus 20 sending an IR interrogation signal to the IR-based wall unit in response to a brake being activated on the patient support apparatus, a power cord of the patient support apparatus 20 being plugged into an electrical outlet, and/or in response to another condition.

It will also be understood that, although patient support apparatus 20 has been primarily described as having either a single UWB transceiver or four UWB transceivers (e.g. FIG. 14), patient support apparatus 20 may have different numbers of UWB transceivers in different embodiments, including embodiments with two UWB transceivers 124, three UWB transceivers, or more than four UWB transceivers 124.

Additionally, in some embodiments, patient support apparatus 20 may be adapted to display on one or more of its own displays—which may be included within any of control panels 54—its location as determined by its communication with one or more wall units 60. Additionally, or alternatively, patient support apparatus 20 may also be configured to display on its own display any medical devices 166 that it has determined to be positioned within space volume 168.

It will also be understood that, although the foregoing description has been made primarily with respect to one or more patient support apparatuses 20 that incorporate one or more UWB transceivers 124 therein for communicating with one or more wall units 60, the incorporation of such UWB transceivers 124 may also or alternatively be incorporated into one or more thermal control units that are used to control the patient's temperature. In such embodiments, the thermal control units include one or more UWB transceivers 124 (and, in some cases, one or more BT transceivers 122 and/or other types of transceivers) that communicate with one or more wall units 60 in order for the location of the thermal control units to be determined. In some of these embodiments, the thermal control units include a network transceiver, which may be the same as network transceiver 94 of patient support apparatus 20, and which enables them to report their location to a server on network 80, such as, but not limited to, patient support apparatus server 84. The thermal control units may be the same type of thermal control units that are disclosed in commonly assigned U.S. Pat. No. 10,390,992 issued Aug. 27, 2019, to inventors Christopher J. Hopper et al, and entitled THERMAL CONTROL SYSTEM, the complete disclosure of which is incorporated herein by reference. Additionally, or alternatively, the thermal control units may include any of the same structural or functional characteristics of any of the thermal control units disclosed in the following patent references: U.S. patent publication 2020/0179161 filed Dec. 11, 2019, by inventors Andrew M. Bentz et al, and entitled THERMAL SYSTEM WITH THERMAL PAD FILTERS; U.S. patent publication 2019/0269547 filed Mar. 4, 2019, by inventors Gregory S. Taylor et al, and entitled THERMAL SYSTEM WITH STEP RESPONSE; U.S. patent publication 2019/0231938 filed Jan. 31, 2019, by inventors Ryan Ariel Alvarez et al, and entitled THERMAL SYSTEM WITH DRAINAGE BAG; U.S. patent publication 2019/0192339 filed Dec. 17, 2018, by inventors Gregory S. Taylor et al, and entitled THERMAL SYSTEM WITH GRAPHICAL USER INTERFACE; U.S. patent publication 2019/0192338 filed Dec. 13, 2018 by inventors Gregory S. Taylor and entitled THERMAL SYSTEM WITH OVERSHOOT REDUCTION; U.S. patent publication 2019/0125580 filed Oct. 24, 2018, by inventors Gregory S. Taylor et al, and entitled THERMAL SYSTEM WITH MEDICATION INTERACTION; and U.S. patent publication 2018/0214301 filed Jan. 26, 2018, by inventors Erika Grace Fojtik et al, and entitled THERMAL CONTROL SYSTEM WITH FLUID CARTRIDGES, the complete disclosures of all of which are incorporated herein by reference.

It will also be understood that, in some embodiments, patient support apparatus 20 may include one or more UWB transceivers 124 that are positioned on movable components of patient support apparatus 20, such as, but not limited to, litter frame 28 and/or siderails 36. In such embodiments, controller 132 is apprised of the current location of each moveable transceiver 124 by way of one or more sensors that measure the current location of the one or moveable components. Controller 132 then uses this current location information when it communicates with one or more wall units 60 and/or with one or more tagged medical devices 166.

In some embodiments, linked wall units 60a and/or unlinked wall units 60b may include additional information stored therein that is shared with patient support apparatus 20 when patient support apparatus 20 is positioned within range of the wall unit 60. Such additional information may include location information identifying the relative position of the wall unit 60 with respect to one or more other wall units 60 that are positioned nearby. Additionally or alternatively, the wall units 60 may include information regarding the thickness and/or materials of the wall 62 to which it is attached, wherein such information provides an indication to the patient support apparatus 20 of the amount of attenuation that UWB signals will likely experience when traveling through that wall. Additionally or alternatively, the wall units 60 may include information identifying their general location within the healthcare facility (e.g. room 400, bay A of room 302, hallway X, maintenance area Y, radiology department, emergency department, etc.) and/or information identifying a more specific location of the wall units 60 within the healthcare facility (e.g. a set of X, Y, Z coordinates in a frame of reference that includes all, or a portion of, the healthcare facility; a height on the wall 62, a distance from one or more landmarks and/or architectural features within the healthcare facility, and/or other more specific information). In some embodiments, patient support apparatus 20 is adapted to utilize this information to determine its location within the healthcare and/or to determine whether it is positioned on the same side of the wall 62 as a particular wall unit 60. The aforementioned information may be included within the wall unit, either in addition to, or in lieu of, any of the previously discussed information that may be stored in the wall unit (e.g. the direction in which the wall unit 60 is facing, the wall unit ID, the dimension(s) of the threshold ranges 158 and/or 160, etc.).

It will be understood that, although the majority of the above-disclosure has discussed the use of transceivers 104 and 124 for determining the distances between themselves and/or one or more tags, transceivers 104 and/or 124 may be additionally and/or alternatively used for determining angular information between themselves and/or one or more tags. Thus, in some embodiments, transceivers 104 and 124 may be used to not only determine how far each transceiver 124 is positioned from a nearby transceiver 104, but also the angular relationship of each transceiver 124 relative to the nearby transceiver 104.

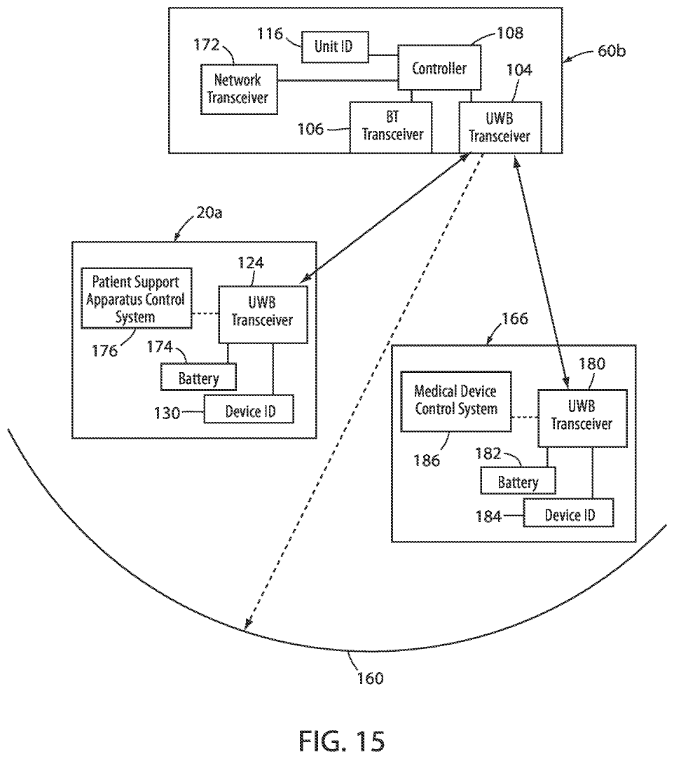

FIG. 15 illustrates an alternative embodiment of an unlinked wall unit 60b. The unlinked wall unit 60b of FIG. 15 differs from the unlinked wall unit 60b of FIG. 7 in that it includes a network transceiver 172. Network transceiver 172 allows unlinked wall unit 60b to communicate with one or more of the access points 82, and thus with local area network 80. As will be discussed, unlinked wall unit 60b may be configured to forward information about medical devices that it detects within its vicinity (e.g. within a threshold distance 160) to patient support apparatus server 84 and/or to another server on network 80. Network transceiver 172 may be the same as network transceiver 94 of patient support apparatus 20, or it may be another type of network transceiver.

Unlinked wall units 60b of the type shown in FIG. 15 are adapted to use their UWB transceiver 104 to determine how far away any medical devices 166 and/or patient support apparatuses 20 or 20a that are positioned within communication range are located. If one or more medical devices 166 and/or patient support apparatuses 20, 20a are positioned within threshold range 160, controller 108 of wall unit 60b is configured to send a message to patient support apparatus server 84 via network transceiver 172. The message informs patient support apparatus server 84 of the location of the medical devices 166 and/or patient support apparatuses. The location corresponds to the location of wall unit 60b, which, as noted previously, was determined when initially installed (such as during a surveying operation) and which is recorded in a memory accessible to server 84.

In the example shown in FIG. 15, a patient support apparatus 20a is depicted that is modified from the patient support apparatuses 20 previously described. Patient support apparatus 20a differs from patient support apparatuses 20 in that it includes a battery 174 that is adapted to provide power to UWB transceiver 124 independently of the main power source for patient support apparatus 20. In other words, if patient support apparatus 20a is placed in a storage room and its power is turned off, or is placed somewhere else and its power is shut off by a caregiver or other person, battery 174 is provided so that UWB transceiver 124 is able to operate despite the main power supply of patient support apparatus 20a being turned off. UWB transceiver 124 is therefore still able to communicate with UWB transceiver 104 of wall unit 60b, which allows UWB transceiver 104 to determine the distance of patient support apparatus 20a from itself, even when the patient support apparatus 20a has its power turned off.

UWB transceiver 124 of patient support apparatus 20a is configured to forward patient support apparatus ID 130 to UWB transceiver 104 of wall unit 60b, even when power is turned off on patient support apparatus 20a. By forwarding patient support apparatus ID 130 to wall unit 60b, controller 108 of wall unit 60b is able to determine which specific patient support apparatus 20a it is communicating with. Controller 108 forwards patient support apparatus ID 130 to patient support apparatus server 84 via network transceiver 172 if patient support apparatus 20a is positioned within the threshold distance 160 of wall unit 60b.

Patient support apparatus 20a also includes an onboard control system 176. The onboard control system 176 may comprise the same, or similar, components as shown in patient support apparatuses 20 in FIGS. 5 and 7 (e.g. controller 132, BT sensor 122, network transceiver 94, etc.), or it may comprise a different set of components. In some embodiments, UWB transceiver 124 is communicatively coupled to control system 176, while in other embodiments of patient support apparatus 20a, UWB transceiver 124 may be communicatively decoupled from control system 176. When decoupled, control system 176 may include one or more separate UWB transceivers 124 that are used for determining the location of a medical device 166 relative to patient support apparatus 20a and/or that are used for communicating with wall units 60a and/or 60b when power is supplied to patient support apparatus 20. In those embodiments where UWB transceiver 124 is communicatively decoupled from control system 176, UWB transceiver 124, battery 174, and device ID 130 may be part of a separate UWB tag that is affixed to, or integrated into, patient support apparatus 20a. Such a tag allows wall unit 60b to determine the location of patient support apparatus 20a regardless of whether or not patient support apparatus 20a has its power turned on or not. This can be especially useful for determining the location of patient support apparatuses 20a that are in storage, or otherwise not in use.

Wall unit 60b of FIG. 15 is configured to not only determine if one or more patient support apparatuses 20 and/or 20a are positioned within threshold distance 160, but also whether one or more medical devices 166 are positioned within this distance 160 as well. As shown in FIG. 15, UWB transceiver 104 is configured to communicate with a UWB transceiver 180 integrated into, or attached to, a medical device 166. UWB transceiver 180 is powered by a battery 182 that may be the same as, or similar to, battery 174. Medical device 166 also includes a device ID 184 that UWB transceiver 180 is configured to transmit to wall unit 60b (using UWB transceiver 180). Medical device 166 also includes a control system 186 that, as with patient support apparatus 20a, may be communicatively coupled to, or communicatively decoupled from, UWB transceiver 180, battery 182, and device ID 184. Such decoupling may occur when UWB transceiver 180, battery 182, and device ID 184 are integrated into a UWB tag that is separate from the control system 186 of device 166, or it may occur in other situations.

Whether coupled to, or decoupled from, control system 186, battery 182 is adapted to provide power to UWB transceiver 180 even when power is not supplied to control system 186 (e.g. when the main power of medical device 166 is turned off), thereby enabling UWB transceiver 180 to communicate with UWB transceiver 104 when medical device 166 is turned off. As with patient support apparatuses 20a, this enables wall unit 60b to determine the location of medical devices 166 even when they are positioned in storage areas, and/or in other situations where their power is turned off. If wall unit 60b determines that medical device 166 is positioned within threshold distance 160 of it, it communicates this fact, as well as the device ID 184 of the medical device 166, to patient support apparatus server 84. Patient support apparatus server 84 is therefore able to determine the location of patient support apparatuses 20, 20a and medical devices 166 within a healthcare facility, even when those devices are not currently powered on.

Patient support apparatus server 84 may be configured to share the location information of patient support apparatuses 20, 20a and/or medical devices 166 with authorized personnel associated with the healthcare facility, such as administrators, technicians, service personnel, caregivers, doctors, and/or other individuals. In some embodiments, patient support apparatus server 84 sends this location information to one or more of the electronic devices 96 so that the personnel authorized to use those devices 96 are able to know the location of patient support apparatuses 20, 20a, and/or medical devices 166.

It will be understood that, although FIG. 15 illustrates wall unit 60b communicating with a patient support apparatus 20a, wall unit 60b is also configured to communicate with patient support apparatuses 20. Such patient support apparatuses 20, as have been described herein, however, need to have their main power turned on in order for their UWB transceivers to operate. Accordingly, wall unit 60b of FIG. 15 is configured to communicate with patient support apparatuses 20 when they have their main power turned on, and it is configured to communicate with patient support apparatuses 20a both when they have their main power turned on as well as when they have their main power turned off.

The threshold distance 160 may vary for individual wall unit 60b. Thus, if a wall unit 60b is placed in a large storage area, or other large area, and it is desirable to know if any patient support apparatuses or medical devices are positioned within that large area, wall unit 60b may be configured to use a threshold distance 160 that is large enough to encompass the large area. If wall unit 60b is placed in a hallway, or other location in which a finer granularity of position information is desired, wall unit 60b may use a threshold distance 160 that is smaller. In some embodiments, patient support apparatus server 84 is configured to send to each wall unit 60b the value it is to use for threshold distance 160. Indeed, in some embodiments, wall unit 60b may be configured to use different threshold distances 160 for different patient support apparatuses 20, 20a, and/or for different medical devices 166. Patient support apparatus server 84 may be configured to allow a healthcare administrator, or other authorized personnel, to remotely configure the threshold distances 160 for one or more wall units 60b using their electronic device(s) 96 and/or through other means.

In some embodiments, patient support apparatus server 84 is configured to provide a map, floorplan, or other information to electronic devices 96 indicating the locations of the patient support apparatuses 20, 20a, and/or medical devices 166, as determined by the wall units 60. This location information may be included within a caregiver assistance software application executed by server 84 that communicates with electronic devices 96 and that shares other information about patient support apparatuses 20 with electronic devices 96. For example, the caregiver assistance software application may also share with electronic devices 96 any of the information disclosed in—as well as include any of the features or functions of the caregiver assistance software application disclosed in—any of the following commonly assigned patent applications: U.S. patent application Ser. No. 62/826,097, filed Mar. 29, 2019 by inventors Thomas Durlach et al, and entitled PATIENT CARE SYSTEM; U.S. patent application Ser. No. 16/832,760 filed Mar. 27, 2020, by inventors Thomas Durlach et al, and entitled PATIENT CARE SYSTEM; and/or PCT patent application serial number PCT/US2020/039587 filed Jun. 25, 2020, by inventors Thomas Durlach et al, and entitled CAREGIVER ASSISTANCE SYSTEM, the complete disclosures of which are all incorporated herein by reference.

It will also be understood that, although FIG. 15 illustrates only a single patient support apparatus 20a and a single medical device 166 positioned within threshold distance 160 of wall unit 60b, threshold distance 160 may have a size that enables multiple patient support apparatuses 20, 20a and/or multiple medical devices 166 to be simultaneously positioned within threshold distance 160. It will also be understood that the medical device 166 shown in FIG. 16 may take on a wide variety of forms, including, but not limited to, all of the different forms specifically mentioned herein (e.g. one or more exercise devices, heel care boots, IV stands and/or poles, infusion pumps, DVT pumps, ventilators, patient sensors (e.g. saturated oxygen (SpO₂) sensors, EKG sensors, vital sign sensor, etc.), patient positioning devices (e.g. wedges, turning devices, pumps), ambient sensors (e.g. air temperature, air flow, light, humidity, pressure, altitude, sound/noise), mattresses 42, incontinence pads or one or more sensors adapted to detect patient incontinence, Holter devices adapted to monitor and record a patient's heart signals, patient ID tags or bracelets adapted to be worn by patients, caregiver tags or ID bracelets adapted to be worn by caregivers, pieces of furniture, and/or other types of devices). By sharing location information of these devices and/or patient support apparatuses 20, 20a, patient support apparatus server 84 is not only able to provide status information regarding patient support apparatuses 20, 20a and/or medical devices 166 to remotely positioned personnel via electronic devices 96, but also to assist in inventory management of these devices.

It will also be understood by those skilled in the art that the use of the term “transceiver” throughout this specification is not intended to be limited to devices in which a transmitter and receiver are necessarily within the same housing, or share some circuitry. Instead, the term “transceiver” is used broadly herein to refer to both structures in which circuitry is shared between the transmitter and receiver, and transmitter-receivers in which the transmitter and receiver do not share circuitry and/or a common housing. Thus, the term “transceiver” refers to any device having a transmitter component and a receiver component, regardless of whether the two components are a common entity, separate entities, or have some overlap in their structures.

Various additional alterations and changes beyond those already mentioned herein can be made to the above-described embodiments. This disclosure is presented for illustrative purposes and should not be interpreted as an exhaustive description of all embodiments or to limit the scope of the claims to the specific elements illustrated or described in connection with these embodiments. For example, and without limitation, any individual element(s) of the described embodiments may be replaced by alternative elements that provide substantially similar functionality or otherwise provide adequate operation. This includes, for example, presently known alternative elements, such as those that might be currently known to one skilled in the art, and alternative elements that may be developed in the future, such as those that one skilled in the art might, upon development, recognize as an alternative. Any reference to claim elements in the singular, for example, using the articles “a,” “an,” “the” or “said,” is not to be construed as limiting the element to the singular.

What is claimed is:

1. A patient support apparatus comprising:
a support surface adapted to support a patient;
a first transceiver coupled to a first location on the patient support apparatus, the first transceiver adapted to wirelessly communicate with a wall unit mounted to a wall of a healthcare facility in which the patient support apparatus is positioned;
a second transceiver adapted to communicate with a network of the healthcare facility; and
a controller adapted to use radio frequency (RF) communications between the wall unit and the first transceiver to determine a distance between the wall unit and the first transceiver.

2.-7. (canceled)

8. The patient support apparatus of claim 7 further comprising:
a fourth transceiver coupled to a third location on the patient support apparatus, the fourth transceiver adapted to communicate using RF signals with the wall unit; and
wherein the memory further contains data identifying relative positions of the fourth transceiver with respect to the first and third transceivers.

9. The patient support apparatus of claim 8 wherein the controller is further adapted to determine an orientation of the patient support apparatus with respect to the wall to which the wall unit is mounted.

10. The patient support apparatus of claim 8 wherein the controller is further adapted to use RF communications between the wall unit and the third transceiver to determine a second distance between the wall unit and the third transceiver; and to use RF communications between the wall unit and the fourth transceiver to determine a third distance between the wall unit and the fourth transceiver.

11. The patient support apparatus of claim 8 wherein the first, third, and fourth transceivers are further adapted to communicate with a tag attached to a mobile medical device, and the controller is further adapted to use the first, third, and fourth transceivers to determine a relative position of the mobile medical device with respect to the patient support apparatus.

12. The patient support apparatus of claim 11 wherein the controller is further adapted to associate the mobile medical device with the patient support apparatus if the mobile medical device is positioned within a volume of space defined around the patient support apparatus.

13. The patient support apparatus of claim 1 further comprising an orientation sensor adapted to determine a first geographical orientation of the patient support apparatus, wherein the controller is adapted receive a second geographical orientation of the wall unit and to use the first and second geographical orientations to determine if the patient support apparatus and the wall unit are positioned on a common side of a wall.

14.-15. (canceled)

16. A patient support apparatus comprising:
a support surface adapted to support a patient;
a first transceiver coupled to a first location on the patient support apparatus, the first transceiver adapted to wirelessly communicate with a wall unit mounted to a wall of a healthcare facility in which the patient support apparatus is positioned;
an orientation sensor adapted to determine a first geographical orientation of the patient support apparatus; and
a controller adapted receive a second geographical orientation of the wall unit and to use the first and second geographical orientations to determine if the patient support apparatus and wall unit are positioned on a common side of a wall.

17. The patient support apparatus of claim 16 wherein the controller is adapted to receive the second geographical orientation of the wall unit from the wall unit.

18. The patient support apparatus of claim 16 further comprising a memory in which is stored the second geographical orientation of the wall unit and the controller is adapted to receive the second geographical orientation of the wall unit from the memory.

19. The patient support apparatus of claim 16 wherein the orientation sensor is a magnetometer.

20. The patient support apparatus of claim 16 wherein the first transceiver is an ultra-wideband transceiver.

21. The patient support apparatus of claim 16 further comprising a Bluetooth transceiver adapted to communicate with the wall unit using Bluetooth signals.

22. The patient support apparatus of claim 21 wherein the controller is further adapted to transmit audio signals from a microphone onboard the patient support apparatus to the wall unit using the Bluetooth transceiver.

23. The patient support apparatus of claim 16 wherein the controller is further adapted to use radio frequency (RF) communications between the wall unit and the first transceiver to determine a distance between the wall unit and the first transceiver.

24. The patient support apparatus of claim 23 further comprising a second transceiver adapted to communicate with a network of the healthcare facility, wherein the controller is further adapted to receive a unique identifier from the wall unit and to forward the unique identifier to the network using the second transceiver.

25. The patient support apparatus of claim 23 wherein the controller determines the distance without using any infrared signals.

26. The patient support apparatus of claim 24 wherein the first transceiver is adapted to wirelessly communicate with a second wall unit mounted to a second wall of the healthcare facility, and wherein the controller is further adapted to use RF communications between the second wall unit and the first transceiver to determine a second distance between the second wall unit and the first transceiver.

27. The patient support apparatus of claim 16 wherein the first transceiver is adapted to communicate with the wall unit regardless of an orientation of the patient support apparatus with respect to the wall.

28. The patient support apparatus of claim 24 further comprising:
a third transceiver coupled to a second location on the patient support apparatus, the third transceiver adapted to communicate using RF signals with the wall unit; and
a memory containing data identifying a relative position of the third transceiver with respect to the first transceiver.

29.-102. (canceled)
